(19) United States
(12) Patent Application Publication
(10) Pub. No.: US 2023/0414299 A1
(43) Pub. Date: Dec. 28, 2023

(54) TISSUE INTERFACE SYSTEM

(71) Applicant: Orchard Ultrasound Innovation LLC, Sunnyvale, CA (US)

(72) Inventors: Butrus T. Khuri-Yakub, Palo Alto, CA (US); John N. Irwin, III, Greenwich, CT (US); Andre T. Khoury-Yacoub, Purchase, NY (US); J. Christopher Flaherty, Nottingham, NH (US); R. Maxwell Flaherty, Topsfield, MA (US)

(21) Appl. No.: 18/039,978

(22) PCT Filed: Dec. 16, 2021
(86) PCT No.: PCT/US2021/063743

§ 371 Date: Jun. 2, 2023

Related U.S. Application Data
(60) Provisional application No. 63/286,161, filed on Dec. 6, 2021.
(60) Provisional application No. 63/126,078, filed on Dec. 16, 2020.
(60) Provisional application No. 63/195,292, filed on Jun. 1, 2021.

(51) Int. Cl. A61B 34/00; A61B 18/00

(52) U.S. Cl. CPC A61B 34/25; A61B 18/00; A61B 2017/00199

(57) ABSTRACT

Systems, devices, and method for performing a medical procedure on a patient are provided. A system includes a tissue interface device and a console. The tissue interface device is configured to image tissue and/or deliver energy to tissue. The tissue interface device includes a transmission assembly. The console is configured to transmit energy to the tissue interface device.

### RELATED APPLICATIONS

The present application claims priority to U.S. Provisional Patent Application Ser. No. 63/126,078 (Docket No. USD-003-PR1), entitled “Tissue Interface System”, filed Dec. 16, 2020, which is incorporated herein by reference in its entirety.

The present application claims priority to U.S. Provisional Patent Application Ser. No. 63/195,292 (Docket No. USD-004-PR1), entitled “Tissue Treatment System”, filed Jun. 1, 2021, which is incorporated herein by reference in its entirety.

The present application claims priority to U.S. Provisional Patent Application Ser. No. 63/286,161 (Docket No. USD-008-PR1), entitled “Capacitive Micromachined Ultrasonic Transducer”, filed Dec. 6, 2021, which is incorporated herein by reference in its entirety.

### FIELD OF INVENTIVE CONCEPTS

The embodiments disclosed herein relate generally to systems for performing a medical treatment on a patient, particularly systems that include a treatment component that can navigate along a tissue surface of the patient.

### BACKGROUND

Numerous medical devices involve the transmission of energy into the patient in order to collect image data or treat tissue. There is a need for improved systems, devices, and methods for transmitting energy to diagnose or treat diseases and disorders of patients.

### SUMMARY

According to an aspect of the present inventive concepts, a system for performing a medical procedure on a patient comprises a tissue interface device configured to image tissue of the patient and/or deliver energy to tissue of the patient. The tissue interface device comprises a transmission assembly. The system further comprises a console configured to transmit energy to the tissue interface device. The system is configured to perform a medical procedure on the patient.

In some embodiments, the medical procedure comprises a diagnostic procedure, a therapeutic procedure, or both a diagnostic procedure and a therapeutic procedure.

In some embodiments, the system is configured to ablate target tissue of the patient. The target tissue can comprise tissue selected from the group consisting of: hair follicle or other tissue related to hair; tumor tissue; warts; moles; birth marks; varicose veins; spider veins; prostate tissue; soft palate tissue; tongue tissue; and combinations thereof.

In some embodiments, the system is configured to ablate target tissue of the patient and to avoid damaging non-target tissue of the patient.

In some embodiments, the system is configured to remove a tattoo of the patient.

In some embodiments, the system is configured to remove hair of the patient. The system can be configured to ablate a segment of hair of the patient and/or to ablate the blood supply of a hair.

In some embodiments, the system is configured to promote hair growth.

In some embodiments, the tissue interface device comprises a device selected from the group consisting of: handheld device; catheter; probe; robotically controlled device; and combinations thereof.

In some embodiments, the transmission assembly comprises an array of ultrasound transducers comprising one, two, or more ultrasound transducers. The array of ultrasound transducers can comprise at least one piezo element, at least one CMUT element, and/or at least one piezo element and at least one CMUT element.

In some embodiments, the transmission assembly is configured to deliver HIFU energy to the patient's tissue.

In some embodiments, the system further comprises a spacer configured to be positioned between the transmission assembly and the patient's skin when the transmission assembly is delivering the energy to tissue. The spacer can be configured to cool tissue that is proximate the transmission assembly when the transmission assembly is delivering the energy.

In some embodiments, the system further comprises an algorithm. The algorithm can comprise an artificial intelligence algorithm. The algorithm can be configured to identify target tissue for ablation by the transmission assembly. The algorithm can be configured to differentiate target tissue from non-target tissue. The algorithm can comprise a confirmation routine. The confirmation routine can be configured to allow a clinician to confirm identification of target tissue identified by the system.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a system for treating and/or diagnosing tissue, consistent with the present inventive concepts.

FIG. 2 illustrates a flowchart of a method of treating one or more treatment targets, consistent with the present inventive concepts.

FIGS. 3A-C illustrate various views of an ultrasonic assembly for creating imaging data and/or delivering treatment energy, consistent with the present inventive concepts.

FIGS. 4A-C illustrate various views of another ultrasonic assembly for creating imaging data and/or for delivering treatment energy, consistent with the present inventive concepts.

FIG. 5 illustrates a side view of a device for imaging and/or treating tissue and including an adjustable spacer, consistent with the present inventive concepts.

FIGS. 5A and 5B are each top and side sectional views of a spacer, consistent with the present inventive concepts.

FIG. 6 illustrates a top view of a skin-attached pad for serially locating a device for imaging and/or treating tissue at multiple device locations, consistent with the present inventive concepts.

FIGS. 7A-G illustrate various views of a CMUT transducer and its components, consistent with the present inventive concepts.

FIGS. 8A-B illustrate side views of a flexible transmission assembly in flat and curved geometric states, respectively, consistent with the present inventive concepts.

FIGS. 9A-B illustrate side views of a hinged transmission assembly in flat and curved geometric states, respectively, consistent with the present inventive concepts.

FIG. 10 illustrates a schematic view of a medical system configured to allow a qualified operator to perform a medical procedure from a remote location, consistent with the present inventive concepts.

FIG. 11 illustrates a flow chart of a method for varying energy delivery settings based on the location of target tissue and/or non-target tissue, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

It will be understood that the words “comprising” (and any form of comprising, such as “comprise” and “comprises”), “having” (and any form of having, such as “have” and “has”), “including” (and any form of including, such as “includes” and “include”) and/or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of two or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, “prevention” and the like, where used herein, shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. “Positive pressure” includes pressure above room pressure or simply a pressure that is greater than another pressure, such as a positive differential pressure across a fluid pathway component such as a valve. “Negative pressure” includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component where used herein are the length and diameter, respectively, of the smallest volume hypothetical cylinder which can completely surround the component.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

As used herein, the term “transducer” is to be taken to include any component or combination of components that receives energy or any input and produces an output. For example, a transducer can include an electrode that receives electrical energy and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo and/or CMUT transducer configured to deliver and/or receive ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: heat energy to tissue; cryogenic energy to tissue; electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo and/or CMUT transducer); chemical energy; electromagnetic energy; magnetic energy; and combinations of two or more of these. Alternatively or additionally, a transducer can comprise a mechanism, such as: a valve; a grasping element; an anchoring mechanism; an electrically-activated mechanism; a mechanically-activated mechanism; and/or a thermally activated mechanism.

As used herein, the term “functional element” is to be taken to include one or more elements constructed and arranged to perform a function. A functional element can comprise one or more sensors and/or one or more transducers. In some embodiments, a functional element is configured to deliver energy and/or otherwise treat tissue (e.g. a functional element configured as a treatment element). Alternatively or additionally, a functional element (e.g. comprising one or more sensors) can be configured to record one or more parameters, such as a patient physiologic parameter; a patient anatomical parameter (e.g. a tissue parameter); a patient environment parameter; and/or a system parameter (e.g. temperature and/or pressure within the system). In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a patient anatomical parameter; and combinations of two or more of these. A “functional assembly” can comprise an assembly constructed and arranged to perform a function, such as is described hereabove. In some embodiments, a functional assembly is configured to deliver energy and/or otherwise treat tissue (e.g. a functional assembly configured as a treatment assembly). Alternatively or additionally, a functional assembly can be configured to record one or more parameters, such as a patient physiologic parameter; a patient anatomical parameter; a patient environment parameter; and/or a system parameter. A functional assembly can comprise an expandable assembly. A functional assembly can comprise one or more functional elements.

As used herein, the term “agent” shall include but not be limited to one or more agents selected from the group consisting of: an agent configured to improve and/or maintain the health of a patient; a drug (e.g. a pharmaceutical drug); a hormone; a protein; a protein derivative; a small molecule; an antibody; an antibody derivative; an excipient; a reagent; a buffer; a vitamin; a nutraceutical; and combinations of these.

As used herein, the term “target tissue” comprises one or more volumes of tissue of a patient to be diagnosed and/or treated. Similarly, a “treatment target” or “tissue target” comprises one or more volumes of tissue to be diagnosed and/or treated. “Safety margin tissue” comprises tissue whose treatment (e.g. receiving of ablative energy) yields no significant adverse effect to the patient. “Non-target tissue” comprises tissue that is not intended to receive treatment (e.g. not intended to receive energy). In some embodiments, “target tissue”, “treatment target”, and/or “tissue target” comprises a non-tissue material, such as a pigment particle used in a tattoo, a splinter such as a wood or metal fragment, and/or other undesired material present in a patient's body.

As used herein, the term “system parameter” comprises one or more parameters of the system of the present inventive concepts. A system parameter can comprise energy delivery parameters, such as one, two or more energy delivery parameters selected from the group consisting of: frequency; amplitude; pulse width; duty cycle; area of ultrasound beam; tissue temperature, such as the starting temperature of tissue prior to treatment; and combinations of these. A system parameter can comprise a parameter selected from the group consisting of: an energy delivery parameter; a pressure level; a temperature level; an energy level; a frequency level; an amplitude level; a battery level; and combinations of these. A system parameter can include one or more tissue targets identified to be treated (e.g. ablated), such as tissue targets identified for treatment by an algorithm.

As used herein, the term “patient parameters” comprises one or more parameters associated with the patient. A patient parameter can comprise a patient physiologic parameter, such as a physiologic parameter selected from the group consisting of: temperature (e.g. tissue temperature); pressure such as blood pressure or other body fluid pressure; pH; a blood gas parameter; blood glucose level; hormone level; heart rate; respiration rate; and combinations thereof. Alternatively or additionally, a patient parameter can comprise a patient environment parameter, such as an environment parameter selected from the group consisting of: patient geographic location; temperature; pressure; humidity level; light level; time of day; and combinations of these.

As used herein, the term “image data” comprises data created by one or more imaging devices. Image data can include data related to target tissue, safety margin tissue, and non-target tissue. Image data can also include data related to any implants or other non-tissue objects that are proximate tissue being imaged. Image data can be processed by one or more algorithms of the present inventive concepts, such as to determine one or more locations to treat (e.g. target tissue identified to be ablated or otherwise receive energy), and/or to determine one or more locations to which energy delivery is to be avoided (e.g. non-target tissue). Image data can comprise data produced by a single imaging component, or from multiple imaging components.

As used herein, the term “hair segment” is to be taken to include all or simply a portion of a hair shaft, hair root, hair follicle, and/or hair bulb. Hair segment can include a segment of a blood vessel providing blood to tissue of a hair bulb.

As used herein, the term “transmitting a signal” and its derivatives shall refer to the transmission of power and/or data between two or more components, in any direction.

As used herein, the term “patient use data” shall refer to data related to use of the tissue interface system of the present inventive concepts on a patient (e.g. use of the system in a diagnostic and/or therapeutic procedure performed on a patient). The data can include but is not limited to: operating parameters such as energy delivery parameters, durations of energy delivery; target tissue parameters such as location of target tissue and/or amount of target tissue; patient parameters such as patient physiologic parameters and/or patient location or other patient environment parameters; clinician parameters; clinical site parameters; and combinations of these. Patient use data can include data from multiple patients, such as data collected from multiple patients that interface with one or more systems of the present inventive concepts. In some embodiments, an algorithm of the present inventive concepts uses patient use data from one or more patients to determine a system parameter to be used in performing a medical procedure on a patient.

As used herein, the term “conduit” or “conduits” can refer to an elongate component that can include one or more flexible and/or non-flexible filaments selected from the group consisting of: one, two or more wires or other electrical conductors (e.g. including an outer insulator); one, two or more wave guides; one, two, or more hollow tubes, such as hydraulic, pneumatic, and/or other fluid delivery tubes; one or more optical fibers; one two or more control cables and/or other mechanical linkages; one, two or more flex circuits; and combinations of these. A conduit can include a tube including multiple conduits positioned within the tube. A conduit can be configured to electrically, fluidically, sonically, optically, mechanically, and/or otherwise operably connect one component to another component.

It is an object of the present inventive concepts to provide systems, devices, and methods for performing a medical procedure on a patient. A system can include a tissue interface device and a console. The tissue interface device can be configured to image tissue and/or deliver energy to tissue. The tissue interface device can comprise a transmission assembly, such as a transmission assembly comprising an array of one or more transducers configured to deliver and/or receive ultrasound energy. The console can be configured to transmit energy (e.g. electrical energy) to the tissue interface device.

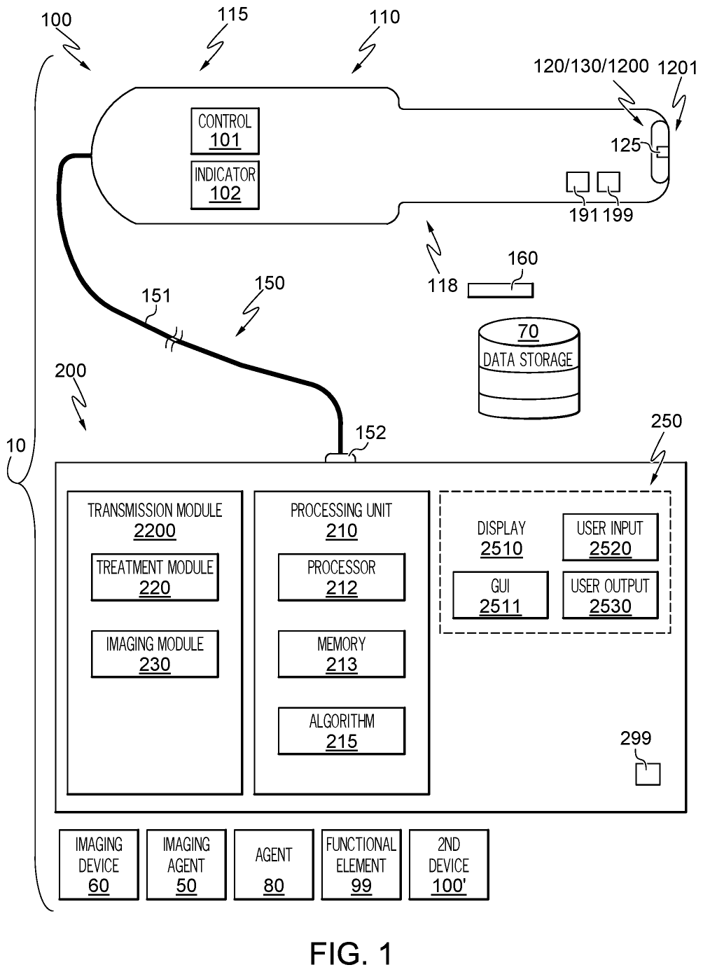

Referring now to FIG. 1, a schematic view of a tissue interface system is illustrated, consistent with the present inventive concepts. System 10 can be configured to perform a medical procedure on tissue, such as a tissue diagnostic procedure, a tissue treatment procedure, or both, such as a procedure that is performed on “target tissue” to be diagnosed and/or treated. In some embodiments, system 10 is configured to deliver energy to target tissue, such as to modify, ablate (e.g. cause necrosis of), and/or otherwise treat the target tissue. Alternatively or additionally, system 10 can be configured to provide image data, image data ID, of tissue and/or one or more objects proximate tissue, such as tissue image data used to provide a diagnosis and/or prognosis (singly or collectively “diagnosis” herein), and/or data used in a tissue treatment procedure (e.g. to guide an ablation and/or other tissue treatment procedure). Image data ID can include image data related to: target tissue; safety margin tissue; non-target tissue; an implant; tattoo pigment; a splinter or other foreign object within tissue; and combinations of these. System 10 includes a tissue interface device, device 100, such as a handheld device as shown that is used to image and/or treat target tissue. Device 100 can comprise a catheter, a probe (e.g. a probe configured to be inserted through a laparoscopic port), and/or a robotically controlled device. In some embodiments, device 100 comprises multiple discrete components. System 10 can further include console 200, which can be configured to operably attach to device 100, such as via connector assembly 150 of device 100 shown. In some embodiments, device 100 comprises all or a portion of console 200.

System 10 can be configured to allow an operator (e.g. a clinician, nurse, technician, and/or other health care provider of the patient) to perform a hair removal procedure in which one or more undesired hairs are “removed” (e.g. caused to eventually be removed while preventing or at least sufficiently delaying subsequent regrowth). For example, device 100 can deliver energy to particular “hair segments” (as defined herein) in a manner configured to thermally ablate (e.g. sufficiently heat to cause necrosis of) or otherwise cause necrosis of the hair segment receiving the energy (e.g. a living part of the hair segment receiving the energy). Alternatively or additionally, system 10 can be configured to promote hair growth, such as by delivering energy to particular target tissue in a manner configured to cause or otherwise enhance the growth of hair. In some embodiments, system 10 is configured to treat multiple hair segments over one or more particular skin surface areas of a patient's body, such as upper lip, forehead, back, thorax, upper arm, forearm, thigh, calf, and combinations of these. In some embodiments, system 10 is configured to create image data ID related to one or more hair follicles and/or other hair segments, and then to deliver energy to the hair follicle and/or other hair segments.

System 10 can be configured to treat target tissue comprising various forms of undesired tissue and/or any tissue whose treatment can provide a benefit. System 10 can be configured to treat target tissue comprising various forms of undesired tissue, such as tumor tissue (cancerous or non-cancerous), warts, moles, birth marks, varicose veins and/or spider veins, and/or other tissue desired to be removed by the patient and/or their clinician. In some embodiments, system 10 is configured to treat target tissue located at a depth below the skin surface, such as a depth of at least 2 mm, or at least 4 mm. In some embodiments, system 10 is configured to treat target tissue located on or otherwise proximate the skin surface. In these embodiments, system 10 can include a spacing element configured to create a gap between an energy delivery element of system 10, and the target tissue, such as is described in reference to spacer 160 herein. In some embodiments, system 10 is configured to treat target tissue comprising cartilage, teeth, and/or bone. For example, system 10 can be configured to deliver energy to a portion of a tooth to treat a cavity (e.g. before the cavity is filled).

System 10 can be configured to treat target tissue such as to remove or at least reduce the presence (“remove” herein) of a tattoo. In some embodiments, the target tissue comprises the pigment of the tattoo, and system 10 is configured to deliver ablation energy to the pigment, such as to cause the pigment to break down and/or otherwise be absorbed by the body. System 10 can be configured to image the pigment and/or to image non-target tissue proximate the pigment, such as to cause energy to be efficiently delivered to the pigment while avoiding adversely affecting non-target tissue proximate the pigment. In some embodiments, system 10 is configured to image at least a portion of a tattoo and enable an operator to select pigment to be treated and pigment to be avoided (e.g. non-target pigment), such that a specified portion of a tattoo can be removed. In some embodiments, device 100 is robotically manipulated, such as is described herein, to enable fine control of device 100, such that fine portions of a tattoo can be removed without damage to and/or removal of surrounding portions. For example, system 10 can be configured to remove pigment in an area with a maximum dimension equal to or less than 2 mm, such as 1 mm, such as 0.5 mm (e.g. to remove a line of pigment as small as 0.5 mm wide). In some embodiments, robotic control of device 100 is based on image data ID.

System 10 can be configured to image and/or ablate tissue of a patient's prostate, such as to diagnose and/or treat benign prostatic hyperplasia (BPH). In some embodiments, system 10 is configured to both image and ablate target tissue of the prostate, as described herein. In some embodiments, system 10 is constructed and arranged as is described in U.S. patent application Ser. No. 17/479,011 (Docket No. USD-001-US-CON1), entitled “Medical Device with CMUT Array and Solid State Cooling, and Associated Methods and Systems”, filed Sep. 20, 2021.

System 10 can be configured to image and/or ablate tissue of a patient's soft palate and/or tongue, such as to diagnose and/or treat sleep apnea. In some embodiments, system 10 is configured to both image and ablate target tissue of the soft palate and/or tongue, as described herein. In some embodiments, system 10 is constructed and arranged as is described in U.S. Provisional Patent Application Ser. No. 63/195,292 (Docket No. USD-004-PR1), entitled “Tissue Treatment System”, filed Jun. 6, 2021.

System 10 can be configured to diagnose and/or treat one or more types of target tissue selected from the group consisting of: hair segment tissue; tissue proximate a tattoo; prostate tissue; soft palate tissue; tissue of the tongue; tumor tissue; organ tissue; skin; blood; tooth; bone; and combinations of these.

Device 100 can include an assembly for delivering energy to tissue, treatment assembly 120 as shown positioned on functional portion 118 of device 100. Treatment assembly 120 can be configured to treat tissue that is on an external surface of a patient's body (e.g. skin tissue, or tissue underlying skin tissue that has been exposed using surgical techniques). Alternatively or additionally, treatment assembly 120 can be configured to treat tissue that is positioned below other tissue, such as when treatment assembly 120 utilizes energy beam-forming techniques to focus energy below a tissue surface. Treatment assembly 120 can be configured to deliver ultrasound energy, such as high intensity focused ultrasound (HIFU).

Device 100 can include an assembly for producing image data ID, imaging assembly 130 as shown positioned on functional portion 118 of device 100. Imaging assembly 130 can produce image data ID that is provided to an operator of system and/or used by system 10 in a medical procedure (e.g. used in a tissue treatment procedure performed in a closed loop mode, in an automated mode, and/or in a semi-automated mode).

In some embodiments, imaging assembly 130 and treatment assembly 120 comprise the same assembly, transmission assembly 1200 (e.g. a combination of imaging assembly 130 and treatment assembly 120). For example, transmission assembly 1200 can comprise an array of ultrasound elements configured to both produce tissue image data ID as well as deliver energy to tissue. In some embodiments, transmission assembly 1200 is of similar construction and arrangement as transmission assemblies 1200′ and/or 1200″ described herebelow.

Treatment assembly 120, imaging assembly 130, and/or transmission assembly 1200 (singly or collectively “transmission assembly 1200 or “assembly 1200) can comprise one, two, or more elements, elements 125 shown, each element configured to deliver and/or receive one or more forms of energy. Elements 125 can be configured to transmit and/or receive one or more forms of energy selected from the group consisting of: ultrasound energy; electromagnetic energy; light energy; mechanical energy (e.g. vibration); chemical energy; and combinations of these. Elements 125 can comprise multiple elements 125 positioned in an array, such as a two-dimensional (2D) or three-dimensional (3D) array.

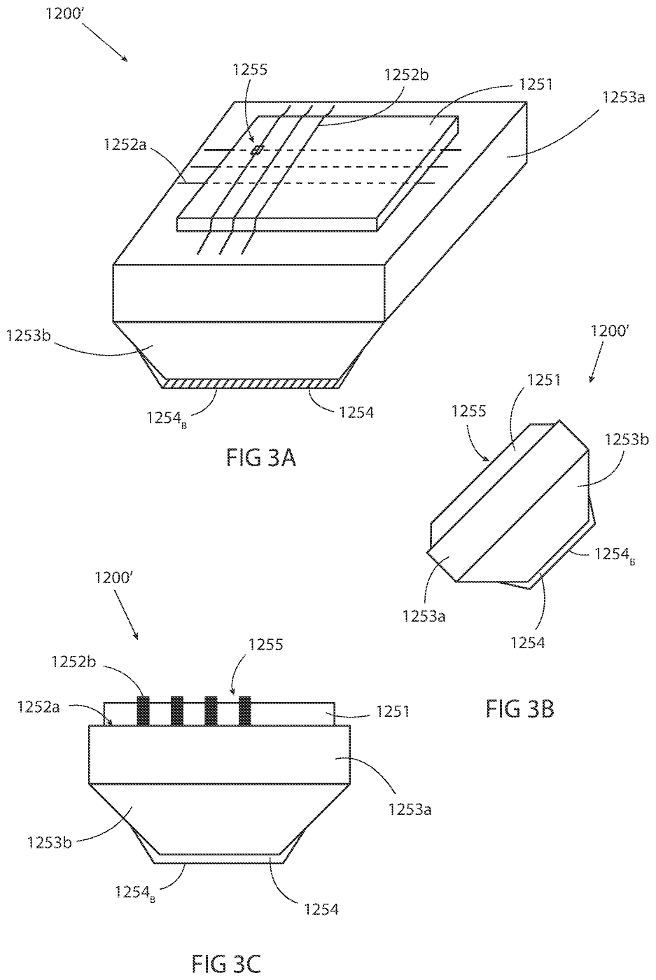

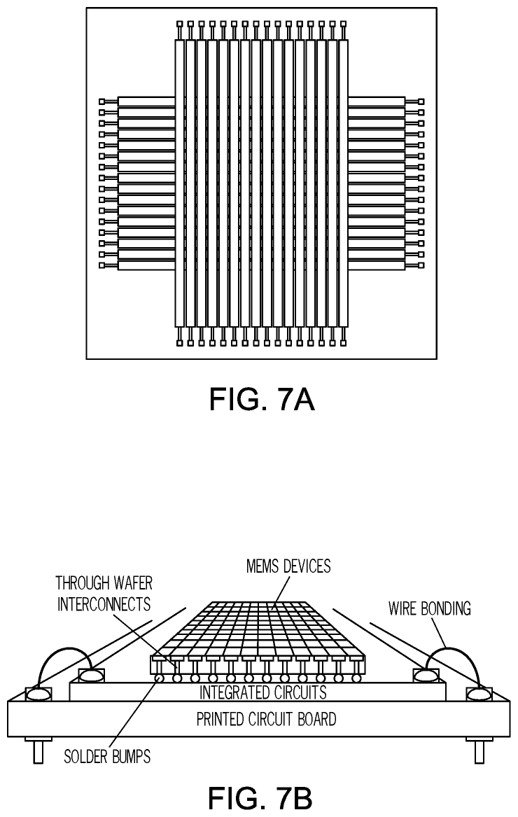

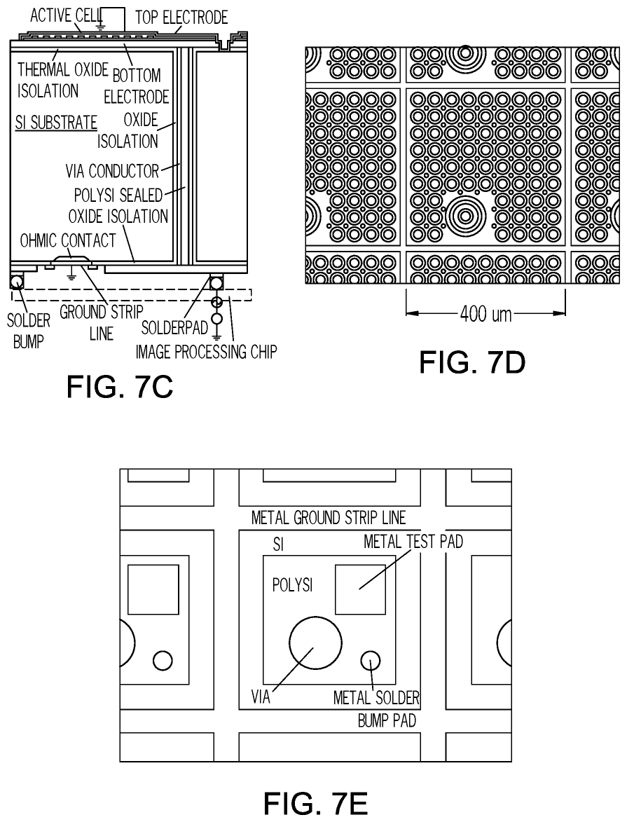

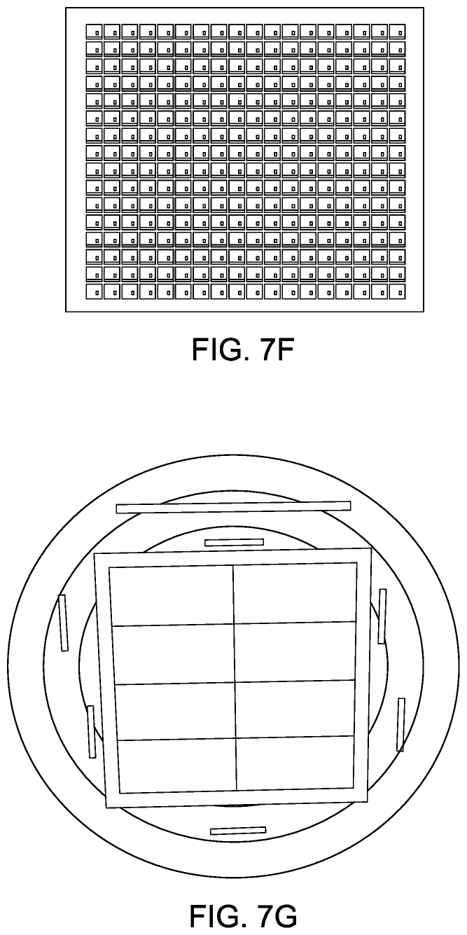

Transmission assembly 1200 can comprise one, two, or more elements 125 configured to deliver ultrasound energy (e.g. deliver ultrasound energy to ablate and/or otherwise treat target tissue). Alternatively or additionally, elements 125 can comprise one, two, or more elements 125 configured to receive ultrasound energy (e.g. receive ultrasonic reflections from target tissue and/or locations proximate target tissue). Elements 125 can comprise one, two, or more piezo ultrasound transducers, and/or one or more capacitive micromachined ultrasonic transducers (CMUTs). In some embodiments, elements 125 comprise one, two, or more piezo transducers as described herein in reference to FIGS. 3A-C and/or 4A-C. In some embodiments, elements 125 comprise one, two, or more CMUT elements as described herein in reference to FIGS. 7A-G. Capacitive micromachined ultrasonic transducers (CMUTs) operate on principles different from those of piezoelectric transducers. Piezoelectric transducers are based on piezoelectric crystals that bend or contract/expand in response to applied electric fields. A CMUT has a cavity formed in a substrate (e.g. a silicon-based substrate). A thin membrane equipped with an electrode can be suspended atop of the cavity. Another electrode can be positioned below the cavity, fixed on a substrate. When a voltage is applied between the two electrodes, electrostatic attractive forces pull the membrane downwards, shrinking the cavity. When the voltage drop is removed, the membrane rebounds. If the applied voltage is a sinusoid at sufficiently high frequency, the membrane vibrates at the same frequency and sends acoustic energy into the medium with which it is in contact. As opposed to piezoelectric transducers, CMUTs have no significant internal loss mechanism and essentially lack self-heating.

In some embodiments, elements 125 are configured to deliver focused ultrasound, such as HIFU. In these embodiments, elements 125 can comprise an array (e.g. a 1D or 2D array) of multiple ultrasound transducers (e.g. piezo and/or CMUT transducers) that delivery one, two or more “HIFU beams”, such as to deliver HIFU to multiple treatment targets simultaneously. Two or more HIFU beams can be delivered from one, two, or more transmission assemblies 1200. Transmission assembly 1200 can be configured to deliver ultrasound energy (e.g. HIFU) to treatment targets located at varying depths from the skin surface, and various distances from assembly 1200.

In some embodiments, transmission assembly 1200 can comprise one, two, or more sets of arrays of ultrasound-based elements 125 (e.g. in a tiled arrangement), such as when each array is configured to deliver one, two, or more HIFU beams. In these embodiments, procedure time can be significantly reduced (e.g. procedures covering large numbers of treatment targets and/or large volumes of any amount of treatment targets). In some embodiments, transmission assembly 1200 directs multiple HIFU beams to different treatment targets. In some embodiments, system 10 is configured to identify the location of multiple treatment targets (e.g. hair segments identified in image data ID) and to classify these locations in a table of registration data (e.g. various registration data RD in a grid format). Once device 100 is in place (e.g. positioned with functional portion 118 proximate the tissue targets), multiple HIFU beams can be delivered via transmission assembly 1200 to ablate or otherwise treat these tissue targets. In some embodiments, treatment of the tissue targets is arranged such that neighboring tissue targets do not receive energy simultaneously (e.g. to avoid undesired heating of non-target tissue). In some embodiments, the delivery of HIFU energy from one or more HIFU beams is arranged such that there is a time delay between delivery of energy to a first tissue target and a neighboring tissue target (e.g. similarly to avoid undesired heating of non-target tissue).

Transmission assembly 1200 can comprise multiple discrete transmission assemblies 1200 (e.g. each containing one or more elements 125), that can be independently placed at corresponding multiple device locations DL (e.g. to image or deliver energy to multiple associated volumes of tissue simultaneously or sequentially).

In some embodiments, at least a portion of transmission assembly 1200 allows light to pass through the portion, such as light passing through a “window portion” of transmission assembly 1200. In these embodiments, a light-based imaging device (e.g. a light-based imaging assembly 130 and/or imaging device 60) can deliver and/or receive light to a target location via the window portion. In some embodiments, transmission assembly 1200 comprises such a window portion as is described in U.S. Pat. No. 10,592,718. In some embodiments, a functional element 199, described herebelow, can comprise a sensor configured to measure a physiologic parameter of the patient is positioned behind a window portion of transmission assembly 1200, such as a functional element 199 comprising an optical sensor configured to measure a physiologic parameter of the patient by transmitting and/or receiving light through the window portion.

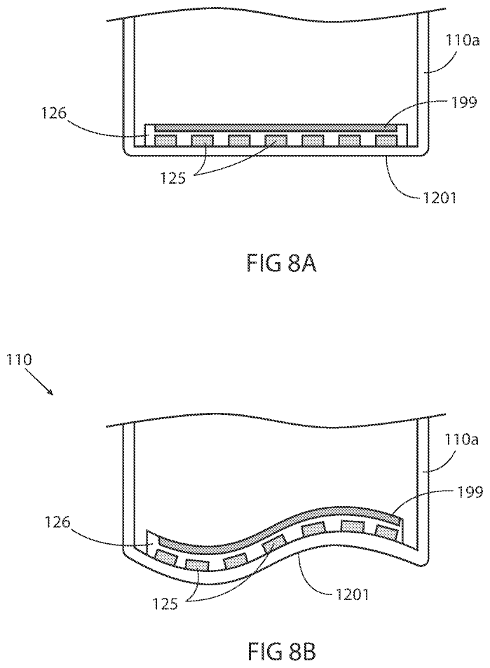

In some embodiments, transmission assembly 1200 comprises a surface (e.g. surface 1201) that comprises a non-linear shape, and/or is configured to transition between a linear shape (e.g. a single plane geometry) and/or a curvilinear shape (e.g. a multiple plane geometry), such as is described herein in reference to FIGS. 8A-B and/or 9A-B. In these embodiments, elements 125 can comprise a similar non-linear shape (e.g. permanent or adjustable), also as described herein.

During a single clinical procedure, transmission assembly 1200 can be positioned at one or more different locations (e.g. one or more different locations on the patient's skin), each a device location DL. From each location DL, transmission assembly 1200 can produce image data ID (e.g. via imaging assembly 130), and/or deliver energy (e.g. via transmission assembly 1200).

In some embodiments, elements 125 comprise one or more elements configured to deliver and/or receive ultrasound energy, such as piezo elements (e.g. as described herebelow in reference to FIGS. 3A-C and/or 4A-C) and/or capacitive micromachines ultrasonic transducer (CMUT) elements (e.g. as described herebelow in reference to FIGS. 7A-G. Transmission assembly 1200 can be configured to deliver ultrasound energy at various frequencies, such as frequencies between 20 MHz and 100 MHz (e.g. for imaging where device 100 would produce image data from tissue locations from 3.3 cm below the skin surface to about 0.66 cm below the surface). In some embodiments, device 100 is configured to also deliver HIFU energy (e.g. to ablate target tissue), and system 10 can use the same frequency to image and ablate (e.g. and the treatment zone would also correspond to the depth between 3.3 cm and 0.66 cm below the skin surface). In some embodiments, transmission assembly 1200 is configured to focus ultrasound delivery at a depth of at least 3.3 cm, or at least 0.66 cm below a tissue surface. In some embodiments, elements 125 comprise one, two, or more ultrasound elements, and at least one element configured to deliver and/or receive another form of energy. In some embodiments, transmission assembly 1200 is configured to image and/or treat tissue from the surface of the skin to about 6 cm below the surface of the skin, such as to about 3.3 cm below the surface of the skin. In some embodiments, an algorithm of system 10 (e.g. algorithm 215 described herein comprising an AI algorithm) is configured to adjust the frequency of the imaging and/or treatment ultrasound energy delivered by transmission assembly 1200 to adjust the depth of imaging and/or ablation. In some embodiments, the frequency of the ultrasound energy delivered can be adjusted based on the proximity of target tissue to non-target tissue, for example, when non-target tissue is between transmission assembly 1200 and target tissue, and/or when non-target tissue is behind (e.g. deeper than) target tissue. Additionally or alternatively, the frequency of the ultrasound energy delivered can be adjusted based on the depth of target tissue (e.g. the distance of the target tissue from transmission assembly 1200 when transmission assembly 1200 is positioned against the skin of the patient).

Transmission assembly 1200 can comprise an array of transmission elements 125 comprising ultrasound transducers (e.g. piezo and/or CMUT ultrasound transducers). The aperture of the array and the number of elements 125 in the array, as well as the frequency, set the field of the view of device 100 and its resolution. As an example, with a transmission assembly 1200 including an array of elements 125 that is 1 cm by 1 cm on the side, device 100 will image a conical volume that emanates away from the array at an angle of 45 degrees from the normal, and extend in depth to roughly 3.3 cm when operating at 20 MHz, and a depth of 0.66 cm when operating at 100 MHz. The penetration depth can be increased by using special excitation methods such as coded excitation and then match filtering the received signal to enhance the signal to noise ratio and thus increase the penetration depth. Device 100 can address the elements 125 in an X-Y fashion or as individual elements. In order to keep the number of electronic channels limited in quantity, device 100 can be configured to perform X-Y addressing of the elements 125. In some embodiments, device 100 is configured to perform full addressing (e.g. individual addressing of the elements 125), such as to provide advantages in the control of imaging operation. The imaging resolution provided by transmission assembly 1200 is related to the size of the aperture, frequency, and location of the focus in depth away from the array of elements 125. As an example, operating at 20 MHz, the wavelength of the ultrasound in tissue is 0.075 mm and the imaging resolution would be about 0.075 mm with an array of elements 125 that is 1 cm on the side and while focusing at a distance of 1 cm below the surface. If an array of elements 125 of the same size is operating at 100 MHz, the wavelength of the ultrasound wave would be 0.015 mm and similarly, the imaging resolution would be 0.015 mm.

Transmission assembly 1200 can be configured to deliver ultrasound energy in the form of continuous, or quasi-continuous, waves with a duration up to several seconds. Device 100 can be configured to minimize the duration of time associated with treating one or more volumes of target tissue. Transmission assembly 1200 can be configured to deliver high intensity focused ultrasound (HIFU) in the several 100 Watts/cm 2 or several MegaPascals of pressure range. For example, a pressure of 1 MPa corresponds to a power density of 30 W/cm², a pressure of 2 MPa corresponds to 120 W/cm², and so on. When operating at high frequency, where the focal spot size is about one wavelength (0.075 mm at and 0.015 mm at 100 MHz) correlates to only 5.6 mW of power at focus to achieve a power density of 100 Watts/cm² at 20 MHz, and 0.225 mW to achieve the same 100 Watts/cm² at 100 MHz. If the pulse duration delivered by device 100 at 100 Watts/cm² is 150 msec, then that would correspond to an energy of 0.84 mJ for operation at 20 MHz, and 0.033 mJ for operating at 100 MHz. Based on the target tissue type (e.g. a hair follicle or other hair segment) and/or location, device 100 is configured to deliver sufficient output pressure that is capable of generating the necessary pressure at focus (e.g. at the location of the target tissue). Simulations of the transmission assembly 1200 (e.g. of FIGS. 3A-C and/or 4A-C) performance predict an output pressure of over 10 kPa/V, hence a 100V excitation should generate 1 MPa on the surface 1201 of the transmission assembly 1200, and with a focusing gain (due to focusing the pressure from many elements 125 in an array), it is relatively easy to deliver up to several MPa which is more than enough to deliver ablation. In some embodiments, transmission assembly 1200 is configured to deliver at least 1 MPa to target tissue, such as at least 2 MPa, at least 3 MPa, at least 4 MPa, and/or at least 5 MPa.

Once positioned on the patient's tissue at a device location DL (e.g. the patient's skin or tissue of an open surgical site), transmission assembly 1200 is capable of delivering energy to (e.g. deliver ablative energy to) a particular volume of tissue proximate location DL, referred to herein as the available treatment volume, or ATV. In some embodiments, the volume of tissue that is imageable by transmission assembly 1200 includes at least the ATV. The ATV is based on the design of transmission assembly 1200, as well as the properties of the tissue proximate device location DL. In some embodiments, the ATV is determined based on image data ID created by transmission assembly 1200 (e.g. image data comprising density and/or other properties of tissue proximate transmission assembly 1200).

Transmission assembly 1200 can be constructed and arranged as described herebelow in reference to FIGS. 3A-C, 4A-C, and/or 7A-G. As described in these figures, transmission assembly 1200 can be configured to deliver ultrasound energy in frequency ranges between 25 MHz and 100 MHz, such as between 25 MHz and 50 MHz. Transmission assembly 1200 can be configured to both image (e.g. create image data ID representing a volume of tissue) and deliver energy (e.g. HIFU and/or other ultrasound ablative energy) to target tissue (e.g. hair, tumor tissue, and/or other tissue desired to be removed). System 10 can be configured to detect features of the target tissue that can be used to determine whether or not to treat (e.g. ablate), and/or to determine the values of one or more energy delivery parameters of the treatment (e.g. the frequency of ultrasound energy to be delivered). Transmission assembly 1200 can be configured to perform X-Y addressing of elements 125 (e.g. piezo and/or CMUT elements), such as in creating image data ID and/or delivering treatment energy.

Transmission assembly 1200 includes a surface, surface 1201, which can be configured to be positioned on a tissue surface when assembly 1200 is imaging or delivering energy to that tissue surface and/or to tissue proximate the tissue surface. Once positioned, the available treatment volume ATV can be determined (e.g. by algorithm 215 described herein).

Device 100 can include one or more housings, such as housing 110 shown, which can include handle portion 115. Housing 110 can comprise rigid materials and/or flexible materials. In some embodiments, at least a portion of housing 110 is flexible. In some embodiments, system 10 comprises two or more devices 100 with varying housing 110 shapes or configurations, such as a wand-like configuration (as shown), a perpendicular configuration such that handle portion 115 is approximately perpendicular to functional portion 118 (e.g. a “pistol grip” configuration), a palm-held configuration such that handle portion 115 fits in the palm of the operator's hand and functional portion 118 (including transmission assembly 1200) is positioned opposite handle portion 115 from the operator's palm (e.g. device 100 is positioned between the operator's palm and tissue), or other ergonomic configuration configured to allow the operator to comfortably position transmission assembly 1200 proximate target tissue (e.g. for an extended period of time while treating a large tissue area, such as when removing hair from a patient's back). In some embodiments, the operator can select a configuration of device 100 from a kit of devices 100 based on the treatment being performed. Various devices 100 can also include varying transmission assemblies 1200, such as transmission assemblies 1200 of different sizes.

Device 100 can include one or more user input and/or user output components, such as control 101 and/or indicator 102 as shown. Control 101 can comprise one or more buttons, levers, and/or other user input components. Indicator 102 can comprise a light (e.g. an LED), a vibrational transducer, a speaker, and/or other type of user output component. In some embodiments, a display or other portion of user interface 250 is included in device 100, as described herein.

Device 100 can include cooling element 191, which can comprise one or more elements configured to cool at least a portion of device 100 and/or to cool tissue proximate device 100. In some embodiments, cooling element 191 and associated components of system 10 are of similar construction and arrangement to those described in U.S. patent application Ser. No. 16/130,896, titled “Medical Device with CMUT array and Solid State Cooling, and Associated Methods and Systems”, filed Sep. 13, 2018. In some embodiments, cooling element 191 is included in spacer 160 described herein. In some embodiments, cooling element 191 is configured to cool tissue surrounding target tissue (e.g. target tissue and non-target tissue), such as to prevent, or at least limit, damage to non-target tissue proximate target tissue to be ablated. In some embodiments, cooling element 191 is configured to cool (e.g. remove heat from) tissue during a thermal ablation of target tissue, such as when HIFU is delivered to target tissue sufficient to overcome the cooling effect provided by cooling element 191 to thermally ablate the target tissue, while avoiding thermal damage to neighboring non-target tissue.

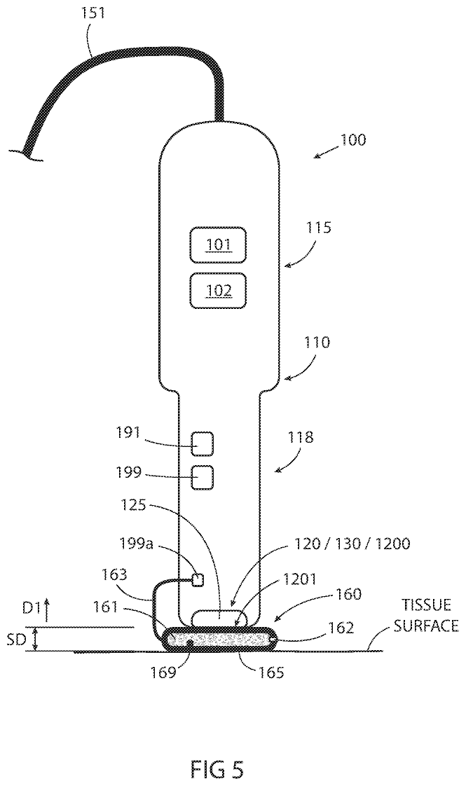

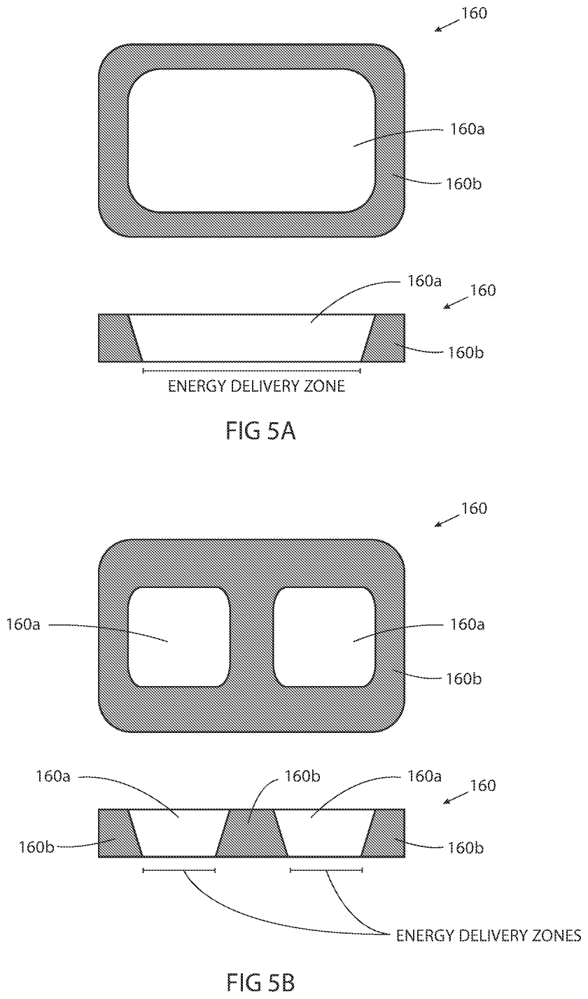

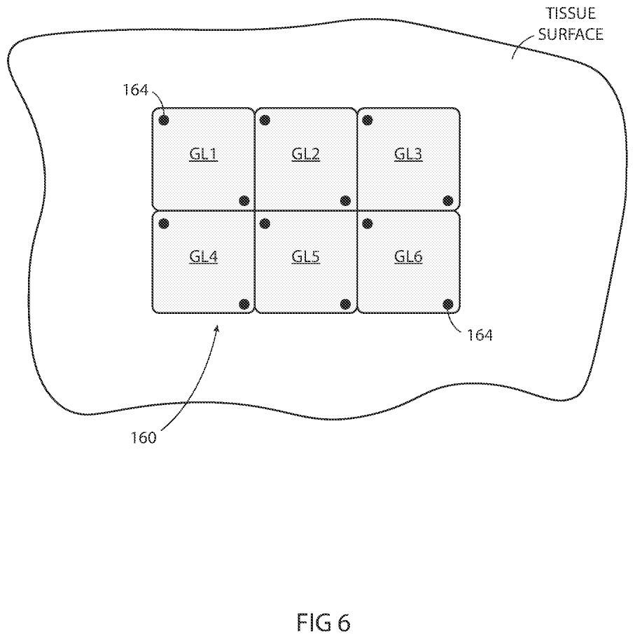

Device 100 can include spacer 160, which can comprise one or more components configured to provide a space between surface 1201 of transmission assembly 1200 and a tissue surface, such as the surface of the patient's skin. In some embodiments, spacer 160 is consistent with the present inventive concepts. as spacer 160 described herebelow in reference to FIGS. 5, 5A and/or 5B. Spacer 160 can comprise a balloon, reservoir, and/or other fluid-containing structure. In some embodiments, spacer 160 comprises water or other sound-conducting material (e.g. with an impedance that approximates the impedance of tissue) such that ultrasound delivered from transmission assembly 1200 passes through spacer 160 and into the patient in a predictable manner. In some embodiments, spacer 160 has an adjustable thickness, such as to allow an operator to adjust (e.g. manually adjust) the distance between surface 1201 of transmission assembly 1200 and a tissue surface, and/or to allow device 100 and/or another component of system 10 to adjust (e.g. automatically adjust) the distance between surface 1201 of transmission assembly 1200 and a tissue surface. System 10 can be configured to automatically adjust the thickness of spacer 160, such as via an AI-based algorithm, and/or such as is described herein in reference to FIG. 5. Spacer 160 can be temporarily or permanently attached to surface 1201. In some embodiments, spacer 160 is configured to removably attach (e.g. adhesively attach) to the patient's skin (e.g. on one side of spacer 160), and/or to removably attach (e.g. adhesively attach) to surface 1201 (e.g. on the opposite site of the spacer). Spacer 160 can comprise a visual grid on a surface, such as to guide attachment of surface 1201 of transmission assembly 1200 at multiple locations, such as is described herein in reference to FIG. 6. In some embodiments, spacer 160 can be configured to provide a cooling function, such as to extract heat from transmission assembly 1200 and/or from tissue of the patient.

In some embodiments, spacer 160 is filled with a fluid (e.g. a recirculating fluid) configured to cool device 100 (e.g. cool transmission assembly 1200) and/or to cool tissue that has been heated using device 100 (e.g. to avoid damage to non-target tissue). In some embodiments, spacer 160 comprises cooling element 191.

Connector assembly 150 can comprise one or more cables, cable 151 shown, which can be configured to operably attach device 100 to console 200 (e.g. electrically, optically, fluidly, mechanically, and/or otherwise operably attach one or more components of device 100 to one or more applicable components of console 200). Cable 151 can comprise one or more elongate elements, such as one or more elements selected from the group consisting of: a wire; an optical fiber; a waveguide; a fluid delivery tube; a pneumatic fluid delivery tube; a hydraulic fluid delivery tube; a mechanical linkage; and combinations of these. Connector assembly 150 can comprise a connector, connector 152 shown, for operably attaching cable 151 to console 200.

In some embodiments, system 10 comprises a second device 100, device 100′ shown, such as a device that is similar to device 100 described herein, but with one or more different features. In some embodiments, console 200 is configured to automatically identify the appropriate model of device 100 and automatically adjust one or more imaging and/or energy delivery settings as appropriate. In some embodiments, system 10 comprises two or more different devices 100 that comprise different transmission assemblies 1200, such as two or more assemblies configured to deliver different forms of energy, and/or to deliver energy in different ways, such as different frequencies, different depths of energy delivery (e.g. depths of focus), different power levels, and/or different volumes of tissue receiving energy.

Console 200 can comprise treatment module 220, a module comprising one or more electronic assemblies for transmitting signals to and/or from treatment assembly 120 of device 100. Treatment module 220 can be configured to deliver energy to an array of elements 125 comprising ultrasound elements (e.g. piezo and/or CMUT elements) that are configured to deliver ultrasound energy (e.g. HIFU or other focused ultrasound energy) to target tissue and/or another target (e.g. an ultrasound energy-activated pharmaceutic drug or other agent delivery device). Alternatively or additionally, treatment module 220 can be configured to deliver another form of energy, such as radiofrequency or other electromagnetic energy (e.g. when elements 125 comprise one or more electrodes); laser and/or other light energy (e.g. when elements 125 comprise a diode and/or a lens); chemical energy; radiation energy; and/or thermal energy (e.g. heat energy and/or cryogenic energy). In some embodiments, treatment module 220 is configured to deliver ultrasound energy and at least one other form of energy.

Console 200 can comprise imaging module 230, a module comprising one or more electronic assemblies for transmitting signals to and/or from imaging assembly 130 of device 100. Imaging module 230 can be configured to transmit and receive signals to and from an array of elements 125 comprising ultrasound elements (e.g. piezo and/or CMUT elements) that are configured to deliver ultrasound energy and/or receive ultrasound energy, such as to collect image data from reflections of ultrasound from tissue and/or implanted objects of the patient. Alternatively or additionally, imaging module 230 can be configured to produce image data via other imaging modalities, such as via delivery and/or receiving of electromagnetic energy; laser and/or other light energy; and/or radiation energy. In some embodiments, imaging module 230 is configured to produce image data based on both ultrasound and another form of energy.

Treatment module 220 and/or imaging module 230, singly or collectively transmission module 2200, can be configured to transmit and/or receive power and/or data, to and/or from, treatment assembly 120 and imaging assembly 130. Transmission assembly 1200 can be configured to transmit and/or receive ultrasound energy (e.g. via an array of elements 125 comprising piezo and/or CMUT elements). Alternatively or additionally, transmission assembly can be configured to transmit and/or receive other forms of energy, such as radiofrequency or other electromagnetic energy (e.g. when elements 125 comprise one or more electrodes); laser and/or other light energy (e.g. when elements 125 comprise a diode and/or a lens); chemical energy; radiation energy; and/or thermal energy (e.g. heat energy and/or cryogenic energy). In some embodiments, transmission assembly 1200 is configured to deliver and/or receive ultrasound energy, and to deliver and/or receive another form of energy.

Console 200 can comprise one or more processing units, processing unit 210 shown. Processing unit 210 can be configured to perform a function selected from the group consisting of: a microcontroller function; a signal-processing function; a data analysis function; a machine learning and/or other artificial intelligence function; and combinations thereof. Processing unit 210 can comprise various electronic componentry, such as: one or more processors, processor 212 shown; one or more memory circuits, memory 213 shown; analog to digital conversion circuitry; digital to analog conversion circuitry; sensor recording and/or drive circuitry; user input interface circuitry (e.g. circuitry to interface with a switch or other user input component); user output interface circuitry (e.g. circuitry to interface with a speaker, light, display, and/or other user output component); and/or other electronic components.

Console 200 can comprise one or more user interfaces, user interface 250 shown. User interface 250 can comprise display 2510 which can include one or more touch screen or other displays. Display 2510 can be configured as a graphical user interface, GUI 2511. User interface 250 can comprise one or more user input components, user input 2520 (e.g. a button, switch, lever, foot pedal, and the like), and one or more user output components, user output 2530, (e.g. an indicator light, a speaker, a tactile transducer, and the like), each as shown. In some embodiments, device 100 includes all or at least a portion of user interface 250 (e.g. handle portion 115 includes a display or other user interface component).

System 10 can include a memory (e.g. memory 213) configured to store instructions for performing one or more algorithms, such as algorithm 215 shown of console 200. Memory 213 can be coupled to processor 212, such that processor 212 can perform algorithm 215. Algorithm 215 can be configured to adjust energy delivery by treatment assembly 120.

Algorithm 215 can be configured to cause transmission assembly 1200 to deliver energy to target tissue, and to at least a portion of safety-margin tissue, while avoiding delivering significant energy to non-target tissue. In some embodiments, the amount of safety-margin tissue can be set by an operator (e.g. via user interface 250). For example, an operator can set a thickness parameter (e.g. 1 mm) to define a volume of safety-margin tissue surrounding each tissue target. In some embodiments, algorithm 215 is biased towards delivering energy to a reduced or otherwise limited volume of target tissue (e.g. avoiding the perimeter of the volume), such as to avoid delivering energy to non-target tissue (e.g. biased towards conservative energy delivery). For example, when target tissue comprises hair (e.g. for hair removal, or other cosmetic procedure), algorithm 215 can be biased towards delivering less energy proximate non-target tissue and/or safety margin tissue (e.g. to provide extra protection for non-target tissue, such as a facial nerve). Alternatively or additionally, algorithm 215 can be biased toward delivering energy to all of a volume of target tissue (e.g. ensuring the perimeter of the volume receives sufficient ablation energy), such as to ablate the entire volume of target tissue (e.g. biased towards aggressive treatment of target tissue). For example, when target tissue comprises a tumor or other diseased tissue to be ablated, algorithm 215 can be biased towards delivering sufficient energy to ablate all target tissue. In some embodiments, the bias of algorithm 215 (e.g. towards a conservative or more aggressive treatment) can be determined by the operator of system 10. In some embodiments, the bias is automatically determined by algorithm 215 based on the location of treatment and/or the therapy being provided (e.g. cosmetic therapy or medical treatment, such as cancer treatment).

In some embodiments, algorithm 215 comprises a learning algorithm, a machine-learning algorithm, and/or an artificial intelligence algorithm (singly or collectively “artificial intelligence algorithm” or “AI algorithm” herein). Algorithm 215 can comprise one or more AI algorithms configured to determine and/or adjust the value of one or more energy delivery parameters and/or other system parameters, such as to adjust the value of one or more system parameters based on image data ID collected by transmission assembly 1200.

Algorithm 215 can comprise an AI algorithm configured to learn during use, such as a learning that occurs based on data collected during a portion of a single clinical procedure (e.g. learning that is used to set a system parameter during that single clinical procedure). Alternatively or additionally, algorithm 215 can comprise an AI algorithm that is configured to learn during multiple clinical procedures (e.g. on the same or different patients), and to use that learning to identify tissue targets for treatment (e.g. at least 5, 10, 20, 30, 50 or 70 tissue targets), to set the value of an energy delivery parameter, and/or to set the value of another system parameter during a subsequent clinical procedure (e.g. on a new patient or a patient that has previously been diagnosed and/or treated using system 10). In some embodiments, system 10 includes at least multiple devices 100 that are configured to share data from multiple clinical sites over the internet or other network, and algorithm 215 is configured to analyze the collected data and provide suggested values for energy delivery parameters for one or more devices 100 based on the analysis. In some embodiments, system comprises an AI algorithm that analyzes patient data based on the clinician that used system 10, where the algorithm provides recommendations and/or other output data on a clinician-by-clinician basis. For example, algorithm 215 can provide a recommended system parameter (e.g. target tissue locations and/or a value for an energy delivery parameter) based on previous parameters or other previous preferences of a particular clinician. Algorithm 215 can provide, such as for a particular clinician, a target tissue location to treat (e.g. near the capsule or near the middle of the prostate in a BPH procedure). Alternatively or additionally, algorithm 215 can provide, such as for a particular clinician, a set of values for energy delivery parameters to use (e.g. larger HIFU or other energy deliveries intending to reduce the overall number of energy deliveries, or smaller HIFU or other energy deliveries intending to reduce the time of each energy delivery).

Algorithm 215 can comprise an AI algorithm configured to identify target tissue, such as an algorithm configured to differentiate target tissue from non-target tissue and/or to differentiate target tissue from non-tissue (e.g. differentiate tissue from an implant).

In some embodiments, algorithm 215 comprises an AI algorithm that is configured to create registration data RD representing anatomical locations of tissue targets (e.g. hair segments, tumor tissue, and/or other tissue), and to provide a suggested “treatment plan”. The treatment plan can include one or more of: an order of tissue targets to be treated; energy delivery settings for the treatment of each tissue target; a volume of tissue proximate each tissue target to receive energy (e.g. ablative ultrasound energy); and/or other treatment parameters. A treatment plan can comprise a plan which minimizes undesired damage to tissue (e.g. thermal damage to target tissue). In some embodiments, algorithm 215 is configured to deliver energy (e.g. ultrasound energy) to treat tissue (e.g. ablate hair segments in a hair removal procedure), while minimizing skin-wrinkling (e.g. skin wrinkling found in today's laser hair removal procedures). Algorithm 215 can be configured to perform a second registration (a “re-registration”), such as if a change in patient position and/or device 100 is detected by system 10. In some embodiments, algorithm 215 is configured to adjust energy delivery during treatment (e.g. ablation) of one or more tissue targets. In some embodiments, system 10 is configured to gather “historical information”, such as information gathered from one or more previous uses of system 10, such as information collected in similar clinical procedures performed using system 10 on different patients, at different clinical sites, and the like. In these embodiments, algorithm 215 can be configured to create a treatment plan based on this historical information.

Algorithm 215 can comprise an AI algorithm configured to identify tissue that has been ablated (e.g. ablated via ultrasound and/or other energy delivered by device 100). For example, algorithm 215 can be configured to differentiate tissue that has been ablated by device 100 versus tissue that has not been ablated (e.g. not been ablated by device 100). Algorithm 215 can comprise an AI algorithm that is configured to provide a set of one or more device locations DL into which device 100 should be placed to treat and/or image a set of multiple discrete target tissue.

Algorithm 215 can comprise an algorithm that is configured to analyze image data ID (e.g. as provided by transmission assembly 1200 and/or imaging device 60), and to identify (e.g. automatically identify) a particular type of target tissue, such as to identify a hair follicle or other hair segment (e.g. based on one or more key features of that type of tissue). Algorithm 215 can be configured to determine the orientation of a tissue target, such as to identify a major axis of a tissue target (e.g. the major axis of a hair segment). Algorithm 215 can comprise an artificial intelligence algorithm configured to identify tissue portions as a particular type of hair segment (e.g. a hair follicle). For example, an operator of system 10 can identify one or more tissue portions as being a hair follicle via an image provided by user interface 250, and algorithm 215 can identify other hair follicles of the patient based on this operator identification. In some embodiments, once identified by algorithm 215, a confirmation routine (e.g. a confirmation routine of algorithm 215) can be performed in which the operator confirms each hair follicle identified by algorithm 215, such that a subsequent ablation of these hair follicles can be performed (e.g. only tissue targets that are confirmed by the operator receive the ablative energy). In some embodiments, the operator can change the status of a hair follicle identified by algorithm 215 as not being a hair follicle. In these embodiments, algorithm 215 can further use artificial intelligence to refine a previous and/or future identification of one or more hair follicles, based on the operator's changing of the classification of the tissue (i.e. the algorithm learns from its mistakes). While the above artificial intelligence-based algorithm 215 has been described in terms of identification (e.g. and subsequent treatment) of hair follicles, other hair segments, and/or other tissue types can be similarly identified by an operator and characterized by algorithm 215. In some embodiments, algorithm 215 is configured to provide an image of the identified tissue of interest (e.g. hair follicle), such as an image that includes indicators or changed graphics (e.g. highlighting) of the tissue of interest on a display of user interface 250. In some embodiments, algorithm 215 is biased towards false negative, such as to not mistakenly identify tissue as a hair follicle. Alternatively, algorithm 215 can be biased towards false positives, such as when all targets identified by algorithm 215 are required by system 10 to be confirmed by the operator before treatment is performed.

Algorithm 215 can comprise an algorithm that is configured to provide image data ID representing one or more tissue targets of interest, such as a hair follicle or other hair segment, to an operator via user interface 250. Using GUI 2511, the operator can mark (e.g. circle) one or more hair segments to be treated (e.g. to receive ablative or other energy). GUI 2511 can be configured to provide a “zoom” function, such as to magnify one or more portions of the image data ID being displayed. In some embodiments, algorithm 215 is configured to segment the image data ID into a grid of cells, such that the operator can confirm proper identification of groups of one or more tissue targets in a cell by cell fashion (e.g. via a confirmation routine of algorithm 215). As described herein, algorithm 215 can comprise an artificial intelligence algorithm configured to automatically identify hair follicles and/or other specific types of tissue to be treated. In some embodiments, GUI 2511 can be configured to indicate (e.g. via a change in color, a surrounding circle, and/or other marking) one or more tissue targets that have received energy (e.g. hair follicles that have been sufficiently ablated), such as to differentiate untreated tissue targets from treated tissue targets. In some embodiments, algorithm 215 characterizes the energy delivered (e.g. via percentage or other quantification) to a tissue target, such as to provide real-time or near real-time (“real-time” herein) status, via GUI 2511, of sufficiency of treatment of a tissue target. In these embodiments, after an operator has treated multiple tissue targets, a repeat procedure can be performed delivering energy to the tissue targets that did not receive sufficient energy to be properly treated (e.g. properly ablated). Algorithm 215 can be configured to automate this type of repeat procedure, such as to automatically deliver energy and/or provide instructions to the operator for the repeated target energy delivery.

Algorithm 215 can be configured to compare a first set of tissue image data ID1 taken at a first point in time T1, to a second set of tissue image data ID2 taken at a second point in time T2, such as to determine if transmission assembly 1200 has changed position from time T1 to time T2. For example, algorithm 215 can be configured to determine that assembly 1200 has translated along tissue (e.g. is at a new device location DL), and/or has tilted (e.g. remains at the same device location DL but an angular change has occurred). If a change in position is detected, and that change is undesirable (e.g. a table of tissue registration data RD is in place and/or energy is currently being delivered), algorithm 215 can be configured to cause system 10 to enter an alert state, such as a state in which energy delivery is stopped, and/or future energy delivery is prevented until the movement issue is resolved.

Algorithm 215 can be configured to cause device 100 to treat multiple tissue targets (e.g. multiple hair follicles or other hair segments) in an automated manner. For example, transmission assembly 1200 can be positioned at a first device location DL1, and multiple tissue targets identified in the ATV (e.g. via algorithm 215 and/or by the operator). In some embodiments, the multiple tissue targets are at least confirmed by the operator, by a confirmation routine of algorithm 215 as described herein. In a subsequent step, algorithm 215 can cause the delivery of energy, in a sequential manner, to each of the tissue targets in the ATV. In some embodiments, tissue targets are treated in a row by row, or column by column manner. In other embodiments, a first tissue target is treated, and a second tissue target that does not “neighbor” the first tissue target is selected for subsequent treatment. This avoidance of energy delivery to neighboring tissue targets is continued, as possible, until all the tissue targets are treated. This avoidance of delivering energy to two neighboring locations can provide the advantage of minimizing heating of non-target tissue, such as to avoid damage to the non-target tissue.

As described hereabove, algorithm 215 can be configured to analyze multiple (e.g. different) sets of tissue image data ID to determine if the position of transmission assembly 1200 has changed relative to a device location DL1 (e.g. device 100 has shifted, tilted, or otherwise been displaced from location DL1). For example, algorithm 215 can analyze the locations of identified tissue targets within the tissue image data ID (e.g. create a table of registration data RD of those locations). If a change in position is detected between two sets of image data (e.g. ID1 and ID2), algorithm 215 can be further configured to determine an offset (e.g. a linear and/or angular offset) between the sets of image data ID. For example, algorithm 215 can be configured to determine which, if any, tissue targets present in the first set of image data ID1 correlate to the tissue targets in the second set of image data ID2. If a percentage (e.g. a percentage above a threshold, such as at least 50%) of the tissue targets in the second set of image data ID2 match the tissue targets of the first set of image data ID1 (e.g. determined based on the location of tissue targets relative to each other), algorithm 215 can determine the amount and/or direction of the offset between the two sets of image data ID (e.g. the amount and/or direction transmission assembly 1200 moved relative to device location DL1). In some embodiments, algorithm 215 determines a set of instructions for the repositioning of transmission assembly 1200 to realign with device location DL1. In some embodiments, the instructions are displayed to the user, such as via GUI 2511. For example, GUI 2511 can display the image data ID with an overlay indicating the current alignment of transmission assembly 1200 relative to DL1, and one or more instructions, for example arrows indicating in which one or more directions to maneuver transmission assembly 1200 to be once again positioned at location DL1 (and in the same angular orientation with respect to the patient). Alternatively or additionally, system 10 can be configured to robotically (partially or fully) reposition transmission assembly 1200 to its original location DL1 (e.g. at its previous angular orientation).

In some embodiments, system 10 is configured, via algorithm 215, to accommodate small movements of transmission assembly 1200 relative to a device location DL, such as to perform a diagnosis and/or a treatment without the operator needing to reposition the assembly 1200. For example, algorithm 215 can be configured to track the relative location of one or more tissue targets within the available treatment volume ATV by analyzing image data ID collected after one or more tissue targets are identified. If transmission assembly 1200 has moved, algorithm 215 can determine the amount of movement, and calibrate the tissue target locations relative to transmission assembly 1200 such that the targets can be ablated without repositioning transmission assembly 1200. System 10 can be configured to accommodate movement of transmission assembly 1200 up to a threshold without needing to be repositioned, for example a movement of no more than 50% of the width of transmission assembly 1200. In some embodiments, the amount of tissue imaged at location DL is greater than the available treatment volume ATV, such that algorithm 215 can track the location of treatment targets that move outside of the available treatment volume ATV.

System 10 can be configured, via algorithm 215, to interleave imaging and treating tissue targets (e.g. as described herein in reference to Step A150 of method A10 herein), for example to confirm transmission assembly 1200 has not moved and the registration of tissue targets to transmission assembly 1200 has not changed. Algorithm 215 (e.g. an AI algorithm) can be configured to analyze image data ID collected to identify movement (or lack of movement) of transmission assembly 1200. In some embodiments, imaging module 230 is configured to continuously collect image data ID during a treatment procedure, via at least a portion of transmission assembly 1200 (e.g. while at least another portion of transmission assembly 1200 is delivering ablative energy to one or more tissue targets). For example, system 10 can be configured to continuously image a first quadrant of the available treatment volume ATV, and to treat tissue targets in a second quadrant of the available treatment volume ATV. Algorithm 215 can be configured to analyze the image data ID and to track the position of one or more treatment targets in the first quadrant. In some embodiments, the analysis of the image data ID in the first quadrant can be interpreted to predict the status of one or more tissue targets in the second quadrant, for example if the targets in the first quadrant are stationary, the targets in the second quadrant can be predicted to be stationary, and system 10 can continue to treat the treatment targets in the second quadrant without reimaging the second quadrant, and without confirming the location of those targets. In some embodiments, the first quadrant comprises four corners of the available treatment volume ATV.

Algorithm 215 can comprise an AI algorithm that is configured to direct two or more HIFU beams, as described herein, to safely and efficiently treat multiple tissue targets (e.g. multiple hair follicles or other hair segments). Transmission assembly 1200 can be configured (e.g. via algorithm 215) to delivery energy to a single location within each tissue target (e.g. to ablate or otherwise treat relatively small volume tissue targets), and/or to deliver energy to multiple locations within a single tissue target (e.g. to ablate or otherwise treat larger volume tissue targets). In some embodiments, transmission assembly 1200 can be configured to deliver multiple energy deliveries to a single location of a tissue target (e.g. to ablate or otherwise treat the tissue target). In some embodiments, algorithm 215 comprises an AI algorithm that is configured to prevent undesired heating of non-target tissue, such as by adjusting the location of and/or timing between repeated energy deliveries (e.g. repeated HIFU or other ultrasound energy deliveries).

System 10 can be configured, via algorithm 215, to create a first set of image data ID1 at a first time, T1, to deliver energy to one or more tissue targets during a first duration of time after T1, and then to create a second set of image data ID2 at a second time, T2. Algorithm 215 can be configured to assess the amount of any tissue swelling that has occurred between time periods T1 and T2. If the amount of swelling is over a threshold, system 10 can be configured to enter an alert state, such as a state in which energy delivery is stopped until the amount of swelling has sufficiently reduced (e.g. as determined by creating a third set of image data ID3 and comparing to the first and/or second sets of image data).

System 10 can be configured, via algorithm 215, to deliver energy (e.g. ultrasound energy) to a set of target locations defining a three-dimensional (3D) space, such as a set of targets in a checkboard pattern but in multiple planes (e.g. at different depths from the skin surface of the patient). The set of target locations can comprise a set of pigments to be broken apart via energy delivery, while minimizing damage to non-target tissue and/or improving healing response of the patient.

In some embodiments, algorithm 215 can be configured to adjust (e.g. intermittently or continuously) the frequency of energy delivery (e.g. ultrasound energy delivery), such as an adjustment performed based on a parameter of the target tissue (e.g. tissue type, tissue location, proximity to non-target tissue, and/or other tissue parameter). In some embodiments, adjustment of frequency or other energy delivery parameter can be accomplished via multiple devices 100 (e.g. two or more devices 100 of dissimilar configurations), In other embodiments, adjustment of frequency or other energy delivery parameter (e.g. ultrasound energy delivery parameter) is accomplished with a single device 100.

In some embodiments, algorithm 215 is configured to deliver energy (e.g. ultrasound energy) to multiple tissue targets in a manner to avoid overheating of non-target tissue. For example, algorithm 215 can cause transmission assembly 1200 to deliver energy to a first tissue target TT1 for a first time period TP1, and then to deliver energy to a second tissue target TT2 for a time period TP2 (e.g. a similar or dissimilar period of time as compared with TP1). Subsequently, the first tissue target TT1 can receive additional energy for a third time period TP3 (e.g. a similar or dissimilar period of time as compared with TP1 or TP2), after which second tissue target TT2 can receive additional energy for a fourth time period TP4. The process can repeat for additional cycles until each of TT1 and TT2 receive sufficient energy for treatment (e.g. sufficient energy to cause tissue necrosis or otherwise be ablated). In some embodiments, three or more tissue targets receive the energy, with similar sequential energy deliveries configured to avoid damage to non-target tissue.

In some embodiments, target tissue to be treated includes one or more hair segments, and algorithm 215 is configured to adjust delivery of energy (e.g. ultrasound energy) based on a hair segment parameter such as: patient race; hair type (e.g. straight, wavy, or curly); hair cross sectional shape (e.g. circular or oval); hair location (e.g. upper lip, back, arm, and/or genital area); and combinations of these.

In some embodiments, target tissue to be treated includes one or more hair segments, and algorithm 215 is configured to determine a hair segment parameter such as described hereabove or otherwise herein. For example, system 10 can be configured to create image data ID (e.g. via imaging assembly 130 and/or imaging device 60) and algorithm 215 can be configured determine the hair segment parameter based on the image data ID.

In some embodiments, algorithm 215 is configured to “suggest” a value for an energy delivery parameter, or an adjustment to an already in place energy delivery parameter value, and system 10 is configured to deliver energy using the suggested value of the parameter only after confirmation by an operator (e.g. a clinician of the patient via a confirmation routine of algorithm 215).

In some embodiments, algorithm 215 comprises a bias, such as a bias towards false positives or false negatives. In some embodiments, algorithm 215 comprises a bias that causes transmission assembly 1200 to deliver energy to target tissue and safety margin tissue, where the safety margin tissue comprises a relatively small thickness, such as when delivery of energy to other than target tissue is to be limited. In some embodiments, algorithm 215 includes a bias that is adjustable by a clinician (e.g. to be based on clinician preference). For example, the bias can be configured to cause the current value of one or more energy delivery parameters to tend towards a set of values selected by (e.g. preferred by) the clinician using system 10. In some embodiments, algorithm 215 comprises a bias that is based on severity of unintended delivery of energy to non-target tissue.

In some embodiments, algorithm 215 comprises a confirmation routine in which an operator of system 10, such as a clinician of the patient, confirms target tissue to be ablated and/or another system parameter. For example, algorithm 215 can comprise an AI or other algorithm that identifies tissue targets to be ablated, and a confirmation routine of algorithm 215 can be performed in which the clinician provides “clinician confirmation information” related to the clinician confirming treatment of all or a subset of the tissue targets should be treated. In some embodiments, the tissue targets identified for treatment by algorithm 215 are provided to the clinician on a display (e.g. via a selectable icon presented on display 2510 and/or other portion of user interface 250), and the clinician approves (e.g. via circling, “clicking on”, and/or otherwise selecting) which of the identified tissue targets should receive treatment.

In some embodiments, system 10 is configured to reduce the size of the patient's tongue, such as to prevent sleep apnea. In these embodiments, system 10 can be configured to treat multiple tissue targets (e.g. multiple discrete volumes of the tongue tissue), such as at least 3, 5, or 10 tissue targets within the tongue. In some embodiments, multiple locations of fatty tissue portions of the tongue are identified for treatment (e.g. ablation), such as when algorithm 215 is configured to analyze image data (e.g. ultrasound-based image data collected by transmission assembly 1200 or other image data collected by transmission assembly 1200 and/or imaging device 60) and determine locations of one or more tissue targets (e.g. fatty tissue targets) to be ablated. In these embodiments, algorithm 215 can be further configured to identify non-tissue targets, such as nerves or other tissue to which thermal damage should be avoided. Algorithm 215 can be configured to treat multiple tissue targets in a particular order (e.g. a non-sequential order in which neighboring tissue targets are not treated sequentially), such as to reduce swelling of the tongue. In some embodiments, system 10 (e.g. via algorithm 215) is configured to treat one side of the tongue in a first procedure, and to treat the opposite side of the tongue in a second procedure that is performed after a minimum time duration (e.g. to prevent difficult breathing due to swelling), such as a minimum duration of at least 1 hour, at least one day, at least one week, or at least one month. In some embodiments, system 10 is configured to track ablated tissue versus non-ablated tissue (e.g. and identify the two tissue types, both as described herein), such as to avoid treating the same tissue of the tongue in both the first and second procedures.

In some embodiments, algorithm 215 is configured to provide one or more potential energy delivery settings for a tissue target, based on the volume and/or other characteristic of that tissue target. The provided energy delivery settings can be used to treat the tissue target, such as after confirmation by a clinician via a confirmation routine of algorithm 215, as described herein.

System 10 can include one or more functional elements, such as functional element 99, functional element 199 (e.g. of device 100), functional element 169 (e.g. of spacer 160) and/or functional element 299 (e.g. of console 200), each as shown.

In some embodiments, functional element 99, 199, 169, and/or 299 comprises one or more sensors, such as one or more sensors configured to measure a patient parameter (e.g. a patient physiologic and/or patient environment parameter), and/or to measure a parameter of a component of system 10.

In some embodiments, functional element 99, 199, 169, and/or 299 comprises one or more transducers, as described herein.

In some embodiments, functional element 99, 199, 169, and/or 299 comprises one or more accelerometers or other sensor configured to detect motion and/or track position of device 100 or other component of system 10.

In some embodiments, functional element 99, 199, 169, and/or 299 comprises one or more batteries, capacitors, and/or other power supplies.

In some embodiments, functional element 99 comprises a marker, such as a pen or other marker configured to mark (e.g. on the skin) one or more treatment targets TT (e.g. to be treated or already treated) on the skin and/or proximate the skin, and/or to mark one or more device locations DL (e.g. from which to deliver energy and/or from which energy has already been delivered).

In some embodiments, functional element 99 comprises a pharmaceutical drug and/or other agent delivery device. For example, functional element 99 can comprise an agent carrying device that is positioned within the patient (e.g. via a clinical procedure and/or ingestion by the patient). The agent carrying device can be configured to prevent administering of the agent into the tissue of the patient (e.g. by surrounding the agent) until energy is delivered to the device (e.g. ultrasound energy is delivered to selected devices, where the ultrasound delivery causes the device to release the agent from the device). Functional element 99 can comprise nanoparticles configured to deliver and/or synthesize a therapeutic agent upon receipt of energy (e.g. ultrasound energy) via device 100. Use of imaging data provided by transmission assembly 1200 or other imaging device of system 10, a subset of functional elements 99 present in the patient's tissue can be located and subsequently activated, such as to provide very localized drug delivery, greatly reducing system effects of the drugs that would result from other forms of delivery. In some embodiments, system 10 is configured to image one or more drug delivery devices that are positioned within the patient (e.g. via an analysis of image data ID by algorithm 215), and to “activate” the drug delivery device via the delivery of energy by transmission assembly 1200 (e.g. delivery of ultrasound energy to an ultrasound energy-activated drug delivery carrier or device).

In some embodiments, functional element 99 and/or spacer 160 comprises an attachment device, such as a rigid or flexible patch that can be placed on the patient's skin, and onto which surface 1201 can be positioned. The patch can include an ultrasonic agent or other imaging agent, such as imaging agent 50 described herein. The patch can include a grid, such as to allow surface 1201 to efficiently and effectively be placed at multiple device locations DL. In some embodiments, transmission assembly 1200 comprises multiple assemblies which can be positioned at multiple device locations DL simultaneously, such as on a single patch-based functional element 99 (e.g. including a grid). Once treatment is complete, the patch can be removed, removing the transmission assemblies 1200 at the same time (e.g. with other portions of device 100 operably connected but at a separate location from the transmission assemblies 1200). The patch can be flexible enough to accommodate patient breathing and other small movements. The pad can have a shape that is configured for placement at particular portions of the patient's anatomy, such as on the skin between the nose and the upper lip.

In some embodiments, functional element 99 comprises a manipulating assembly configured to manipulate (e.g. robotically manipulate) device 100 from one device location DL to another. In some embodiments, functional element 99 comprises a positioning arm configured to adjust and/or maintain the position and/or orientation (position herein) of transmission assembly 1200 (e.g. maintain the position of device 100 at a device location DL), for example, an articulated assembly fixedly attached on one end to a stable surface (e.g. the floor, a work surface, or a patient support such as a bed or chair) and on an opposite end to housing 110 of device 100. The positioning arm can comprise one, two, three, or more articulating joints, each comprising one or more degrees of freedom, such that device 100 can be positioned in 3D space with at least 6 degrees of freedom (e.g. freedom in the x, y, and z directions as well as in the roll, pitch, and yaw of device 100 along a central axis along the length of device 100). The articulation of the positioning arm can be manipulated by the operator, for example, when the joints of the position arm are in a “follow” configuration, such that the joints are free to articulate as device 100 is repositioned (e.g. from a first device location DL1 to a second device location DL2) by the operator. In some embodiments, the articulation of the positioning arm can be locked to maintain the position of device 100. In some embodiments, the positioning arm transitions between a locked configuration and a follow configuration, such that the operator can reposition device 100 in a follow configuration and lock the position of device 100 when transmission assembly 1200 is properly aligned with a treatment area. In some embodiments, the positioning arm transitions from a locked configuration to a follow configuration based on input from the operator, for example, when the operator presses a foot pedal to enable a follow configuration. In some embodiments, the positioning arm is configured in a “zero gravity” mode, such that the positioning arm resists the force of gravity on device 100 (as well as the weight of the positioning arm), such that the position of device 100 is adjustable by the operator and is maintained when the operator stops repositioning device 100 (e.g. the operator “lets go” of device 100). In some embodiments, the positioning arm is robotically controlled. Processing unit 210 can determine a desired position of device 100 (e.g. an optimal position for device 100 to provide treatment to a treatment target as described herein) and control the robotic positioning arm to position device 100 in the desired position.

In some embodiments, functional element 199 comprises a marking assembly configured to mark (e.g. on the skin) one or more treatment targets TT (e.g. to be treated or already treated) on the skin and/or proximate the skin, and/or to mark one or more device locations DL (e.g. from which to deliver energy and/or from which energy has already been delivered). The marking assembly can include a supply of marking fluid (e.g. ink) or other marking material that is dispensed at the location to be marked.

In some embodiments, functional element 199 comprises a locating element configured to register transmission assembly 1200 (e.g. surface 1201) with spacer 160, such as is described herebelow in reference to FIG. 6. For example, a functional element 199 can comprise: a hole or a projection configured to mate with a projection or hole, respectively of spacer 160; a magnet or magnetic material (either or both “magnet” herein) configured to magnetically attract to and align with a magnet of spacer 160; and/or a hook or loop fastening element configured to mate with a loop or hook fastening element, respectively, of spacer 160.

In some embodiments, functional element 199 comprises a thermoelectric cooling element. The thermoelectric cooling element can be mechanically coupled with transmission assembly 1200 (e.g. an array of ultrasound transducers). The thermoelectric cooler can be configured to cool tissue (e.g. non-target tissue), and/or to cool a portion of device 100 (e.g. transmission assembly 1200).

System 10 can comprise imaging agent 50, which can include one or more materials used to operatively couple device 100 to tissue (e.g. one or more materials used to sonically or otherwise operatively couple treatment assembly 120 and/or imaging assembly 130 to tissue). In some embodiments, spacer 160 is filled with and/or otherwise comprises imaging agent 50. In some embodiments, transmission assembly 1200 comprises ultrasound transducers, and agent 50 comprises ultrasound gel or other material configured to enhance coupling of ultrasound transmissions. Imaging agent 50 can comprise a solid material, such as a solid acoustic coupling material (e.g. a material that includes an adhesive to prevent relative movement of transmission assembly 1200 during imaging and/or energy delivery). Imaging agent 50 can comprise a hydrogel, such as a hydrogel compatible to temperatures encountered during tissue ablation (e.g. HIFU tissue ablation on a skin surface or below). In some embodiments, imaging agent 50 comprises a dye or other substance that is configured to change color when exposed to energy delivered by transmission assembly 1200 (e.g. ablative energy, such as ablative ultrasound energy) and/or by a functional element 199 (e.g. positioned proximate transmission assembly 1200) that delivers light (e.g. UV light) or other energy configured to change the color of imaging agent 50. Imaging agent 50 can be positioned (e.g. spread if a gel or placed if a solid) on the patient's back, leg, or other large skin area intended for treatment. In these embodiments, treatment of a tissue within a large surface area of skin, can be tracked (e.g. manually by an operator and/or automatically by system 10 via one or more imaging devices), such as to: track energy delivery from multiple device locations DL until a desired procedure completion is achieved; and/or avoid undesirably treating target tissue multiple times. For example, an operator of system 10 can cause transmission assembly 1200 to deliver energy from multiple device locations DL, each time noting the change in color of agent 50, such as to efficiently and effectively treat the large area (e.g. while avoiding undesired repeating of energy delivery to a single portion of tissue, as described herein). In some embodiments, imaging agent 50 comprises a topical anesthetic (e.g. agent 80 described herein) configured to numb an area prior to treatment by system 10.

System 10 can comprise one or more imaging devices, imaging device 60 shown, which can be used to produce image data, as described herein. Imaging device 60 can comprise one, two or more imaging devices selected from the group consisting of: an ultrasound imaging device; a fluoroscope or other X-ray based imaging device; an MRI, a CT-scanner; a camera such as a visible light camera and/or an infrared camera; a microscope such as an optical microscope; and combinations of these. System 10 can be configured to collect image data ID from imaging device 60, from transmission assembly 1200, or both. Algorithm 215 can be configured (e.g. using AI) to analyze the image data ID, such as to determine tissue targets to be treated and/or to determine the value of an energy delivery parameter and/or other system parameter. In some embodiments, algorithm 215 compares image data received from transmission assembly 1200 (e.g. ultrasound image data) with image data received from imaging device 60 (e.g. an ultrasound imaging device and/or other form of imaging device).

In some embodiments, imaging device 60 comprises an MRI, transmission assembly 1200 includes multiple elements 125 comprising ultrasound transducers (e.g. piezo and/or CMUT ultrasound transducers), and system 10 is configured to collect MRI-based image data ID from imaging device 60, as well as ultrasound-based image data from transmission assembly 1200.

System 10 can comprise data storage 70, which can include one or more memory storage devices, such as to store patient use data, as described hereabove. Patient data and/or other system 10 data can be stored on data storage 70. In some embodiments, these data are used by algorithm 215 to determine the value for an energy delivery parameter and/or other system parameter, as described herein. In some embodiments, one or more clinicians review the data stored on data storage 70 such as to stratify the data and/or to determine outliers in the data. In some embodiments, one or more clinicians review values of system parameters produced by algorithm 215, and reject or confirm the values, such as via a confirmation routine of algorithm 215 as described herein. System 10 can include multiple systems 10, such as when data stored and/or analyzed is provided by multiple consoles 200 and/or multiple devices 100 used with multiple patients over a period of time. In some embodiments, algorithm 215 is configured to determine trends in the data, such as to modify the value of one or more energy delivery parameters and/or other system parameter values. In some embodiments, data storage 70 comprises a portion of “the cloud”, accessible by the manufacturer of system 10, clinicians, patients, and/or other users of system 10.

System 10 can comprise one or more pharmaceutical drugs or other agents as described herein, agent 80. In some embodiments, agent 80 comprises an anesthetic, such as a topical anesthetic configured to at least reduce any pain caused during use of device 100 (e.g. a topical anesthetic deposited at one or more device locations DL). Agent 80 can comprise a drug or other agent that is delivered to the patient (e.g. orally) such as to provide a therapeutic benefit to the patient (such as a drug used to treat a patient condition also to be treated by device 100).

In some embodiments, system 10 is configured to produce photoacoustic-based image data of target tissue (e.g. and neighboring tissue), and to ablate the target tissue by delivering ultrasound energy (e.g. HIFU). For example, system 10 can produce the image data ID using an imaging device 60 comprising a laser configured to deliver light (e.g. multiple wavelength light) that causes the tissue to be imaged to produce ultrasonic emissions (e.g. via heating of the tissue receiving the laser energy). The ultrasonic emissions can be received by a transmission assembly 1200 comprising an array of elements 125 comprising ultrasound transducers (e.g. piezo and/or CMUT transducers, such as are described in reference to FIGS. 3A-C, 4A-C, and/or 7A-G herein). In some embodiments, device 100 is configured in a photoacoustic mode of operation, and transmission assembly 1200 and/or imaging device 60 comprises a laser with multiple wavelength capability (such as an OPO laser), and/or a single or multiple photo-diodes that can be used to illuminate the field of view of an array of ultrasound-based elements 125 of transmission assembly 1200 with either pulsed laser energy, and/or modulated continuous wave energy. For instance, multiple shots of laser light (e.g. at different wavelengths) can be delivered (e.g. by transmission assembly 1200 and/or imaging device 60) to illuminate tissue. The laser light delivery (e.g. nanosecond pulsed laser light) is absorbed by chromophores such as hemoglobin and red blood cells, causing local heating and thermoelastic expansion. Pressure or sound waves are emitted and detected by elements 125 of transmission assembly 1200. The resultant signals produced can be processed by algorithm 215 into high resolution, co-registered ultrasound and photoacoustic images. The image data ID generated by system 10 in this manner can include real-time and/or other data representing location and/or characteristic information of: tissue (including blood oxygen saturation and total hemoglobin of tissue); nanoparticles (e.g. delivered agents or implants); dyes and/or other contrast agents. The image data ID generated can provide drug delivery and/or pharmacokinetic information. The image data ID generated can include information related to the distribution of biomarkers. The image data ID generated can be used to track cells. The image data ID generated can provide real-time co-registration of molecular events (e.g. in reference to the patient's anatomy). In these embodiments, system 10 can be configured to generate a number of images that are cooperatively combined by algorithm 215, such as to highlight different conditions of the tissue (e.g. target tissue). System 10 can produce an image based on a reconstruction of the absorption field, and algorithm 215 can then determine which tissue locations (follicles, other tissue segments, and/or other target tissue as described herein) are to be ablated.

In some embodiments, system 10 is configured to produce ultrasonic-based image data (e.g. as described herein using transmission assembly 1200) of tumor tissue, and also to produce photoacoustic-based image data (e.g. also as described herein) of capillaries surrounding the tumor tissue. In these embodiments, transmission assembly 1200 can be configured to deliver ablative energy (e.g. HIFU or other ultrasonic ablative energy) to the tumor tissue, to the capillaries, or to both. In some embodiments, ablative energy is directed toward a concentration of capillaries (e.g. a concentration of capillaries that may be indicative of an anatomical direction in which the tumor is growing). In some embodiments, system 10 is configured to gather image data ID representing growth of a tumor over time.

In some embodiments, transmission assembly 1200 is configured to deliver HIFU energy to treat undesired conditions of the skin, tissue proximate the skin, and/or hair follicles (e.g. deep-seated hair follicles). The energy delivery performed by device 100 can be guided by the results of photoacoustic imaging, such as photoacoustic imaging carried out with a single laser or multiple laser diodes.

In some embodiments, transmission assembly 1200 is configured to create image data using Doppler ultrasound (e.g. elements 125 comprise piezo and/or CMUT transducers as described herein, and transmission module 2200 is configured to produce Doppler ultrasound-based image data). Using the Doppler data, algorithm 215 can be configured to detect the location of the blood supply to the hair bulb (e.g. regardless of the orientation of the hair or the growth phase), and transmission assembly 1200 can be configured to eliminate (e.g. destroy) this supply of blood to the hair follicle in order to accomplish the removal of the associated hair. Equivalently, system 10 can be configured to perform this approach to destroy the blood supply of tissue having certain skin conditions, such as target tissue comprising a blood supply that is near or below the surface of the tissue.

In some embodiments, system 10 is configured to collect data related to oxyhemoglobin concentration in order to assess (e.g. determine the phase of) hair growth. For example, system 10 can be configured to perform photoacoustic imaging, as described herein, to measure oxyhemoglobin concentration (e.g. of blood proximate the hair follicle), and algorithm 215 can determine the phase of hair growth based on the measurement. Delivery of energy (e.g. ablative energy) by transmission assembly 1200 to particular hair segments (e.g. hair follicles) can be made based on the determined growth phase (e.g. only hair segments in a particular phase receive the ablative energy in the current treatment, such as when hair segments in another phase are scheduled to receive the ablative energy in a subsequent treatment).

In some embodiments, transmission assembly 1200 is configured to deliver a therapeutic configuration of energy to a hair segment, in order to reduce the chance of hair loss, such as by delivering a low amount of energy (e.g. less than an ablative level) that creates a “massaging effect” on the hair (e.g. on the hair follicle). In some embodiments, system 10 is configured to determine the phase of hair growth (e.g. via measurement of oxyhemoglobin concentration as described hereabove), and to deliver the therapeutic configuration of energy (e.g. ultrasound energy), to hairs that are in a particular phase of growth in which the delivered energy has an optimized result. In some embodiments, transmission assembly 1200 is configured to deliver focused energy (e.g. HIFU or other focused ultrasound energy) to cause differential heating of one or more hair follicles versus tissue proximate the one or more hair follicles (e.g. to avoid heating of the scalp when treating hair of the head). In some embodiments, system 10 is configured to deliver energy below a threshold (e.g. below a threshold determined by algorithm 215), such as to avoid ablating or otherwise damaging a hair follicle, while being sufficient to cause a beneficial effect (e.g. an effect such as hair regrowth). In some embodiments, system 10 is configured to deliver energy to promote hair growth in multiple procedures (e.g. multiple procedures performed weeks or months apart). In these embodiments, algorithm 215 can be configured to analyze the effect of a first treatment to determine best system parameters to be used in a second treatment. In some embodiments, agent 80 comprises a drug or other agent configured to further improve hair growth of the patient, such as to further improve growth of hairs treated by energy delivery from device 100.

As described herein, system 10 can be configured to image and ablate tissue of the prostate, such as to treat BPH. In these embodiments, device 100 can comprise a distal portion that is flexible and includes transmission assembly 1200. For example, device 100 can comprise a catheter-based device for insertion through the patient's urethra, such as for positioning transmission assembly 1200 proximate tissue of the patient's prostate tissue to be treated. Algorithm 215 can comprise an AI algorithm configured to analyze image data produced by transmission assembly 1200 and to create registration data RD representing one or more segments of tissue, such as one or more segments of target tissue to be ablated. In some embodiments, a doctor, technician, and/or other clinician of the patient, analyzes registration data, such as to identify one or more target tissue sites to be ablated, and/or to “approve” (e.g. via clinician confirmation information) one or more target tissue sites to be ablated. In some embodiments, one or more key anatomical locations are identified (e.g. via algorithm 215 and/or a clinician) in the registration data RD, such as non-target tissue identified such as to avoid that tissue being damaged during energy delivery to target tissue. Examples of non-target tissues identified (e.g. via an algorithm comprising an AI algorithm) in the registration data can include anatomical locations selected from the group consisting of: the sphincter muscle; a channel in which semen passes thru; median lobe; nerve tissue; and combinations thereof. In some embodiments, algorithm 215 comprises an algorithm (e.g. an AI-based algorithm) that recommends a particular number of discrete ablations to be performed via transmission assembly 1200, such as to efficiently treat the patient's prostate with a minimized number of overall ablations. Algorithm 215 can comprise an algorithm configured to optimize one or more energy delivery and/or other system parameters for ablating tissue of the prostate or other anatomical areas, such as an AI algorithm that determines an optimized energy level (e.g. energy amplitude), energy frequency, energy duty cycle, and/or energy duration. In some embodiments, functional element 199 of device 100 comprises a pressure sensor, and algorithm 215 utilizes recorded pressure sensor information to determine target tissue to be ablated (e.g. where a blockage is likely to be worse). In some embodiments, algorithm 215 comprises an AI algorithm configured to identify tissue of the prostate to be ablated, and prior to ablation of the tissue, a confirmation routine of algorithm 215, as described herein, is performed in which a clinician of the patient approves each target tissue area to receive ablative energy

System 10 can be configured to identify tissue that has been treated (e.g. ablated), such as to properly treat adjacent tissue (e.g. provide a continuous treatment of a volume of tissue), and/or to avoid undesired treating of tissue that has already been treated. In some embodiments, algorithm 215 analyzes image data ID collected by device 100, and the algorithm records tissue that has been treated (e.g. ablated), such as by identifying and recording one or more anatomical landmarks in reference to tissue receiving energy (e.g. and recording the information as image data ID and/or registration data RD). Alternatively or additionally, algorithm 215 can analyze image data ID and identify tissue that has been treated by identifying characteristics of the treatment that can be found in the tissue after treatment (e.g. after ablation). In some embodiments, algorithm 215 comprises an elastographic analysis and/or other tissue characterization analysis to identify treated tissue, or a marker of the treated tissue as described immediately herebelow. In some embodiments, device 100 is configured to “mark” tissue (e.g. permanently and/or temporarily mark tissue), such as to provide one, two, or more markers used to identify tissue that has been ablated or otherwise treated by device 100 (e.g. one or more markers positioned within the treated tissue, on a periphery of treated tissue, and/or at a location proximate the treated tissue). Device 100 can be configured to deliver energy (e.g. HIFU energy) at a particular level (e.g. a higher energy level than the remainder of the treated tissue) that causes an identifiable characteristic in the treated tissue (e.g. an energy delivery that causes cavitation within the treated tissue that can be later identified by system 10). In some embodiments, a clinician using device 100 may create one or more markers in the patient's tissue (e.g. proximate one or more tissue targets), and the clinician (e.g. manually) and/or an algorithm of system 10 (e.g. in an automated or semi-automated fashion) can use the markers to complete a tissue treatment procedure based on the previously created markers.

In some embodiments, algorithm 215 can be configured to differentiate treated tissue from untreated tissue, and/or to differentiate one type of tissue from another type of tissue, based on an elastography measurement performed by system 10. Alternatively or additionally, system 10 can be configured to track energy delivery (e.g. as correlated with image data ID and/or registration data RD) in order to track treated versus untreated tissue. Identifying and/or tracking of treated versus untreated tissue can be very important, particularly when a treatment procedure comprises treatment of a large number (e.g. at least 10, 25, or 50) different tissue targets via individual ablative energy deliveries (e.g. to treat a large number of hair segments or other volumes of tissue). In some embodiments, tissue targets treated or to be treated include hair follicles or other hair segments (e.g. multiple hair segments to be treated in a non-sequential manner) where tracking of treated versus untreated tissue is important to prevent undesired multiple energy deliveries to the same tissue. In some embodiments, tissue targets treated or to be treated include volume portions of the tongue (e.g. ablated in a sleep apnea procedure), where similar tracking of treated versus untreated tissue is desirable. As described herein, system 10 can be configured to create markers in tissue that can be used to track energy delivery, such as by delivering “marking energy” comprising energy (e.g. ultrasound energy) that is delivered at a different set of energy delivery settings than that used to simply ablate tissue (e.g. energy delivered at a higher level than the standard ablative energy, to cause detectable modifications to the tissue that is marked). In some embodiments, system 10 is configured to deliver marking energy in a particular pattern to mark tissue (e.g. two, three or more lines of HIFU or other energy delivery), such as a pattern that can be detected via algorithm 215 using a pattern recognition algorithm. System 10 can be configured to deliver marking energy along various locations of a periphery of a volume of tissue ablated (and/or to be ablated) by system 10, such as to create an identifiable (e.g. via algorithm 215) “treatment boundary”. In some embodiments, algorithm 215 is configured to identify particular tissue types, and/or combinations of tissue types (either or both “tissue types” herein), and to identify these particular tissue types in image data ID and/or registration data RD. Tissue type data TTD can be used in planning a treatment procedure (e.g. defined as reference points) in which certain tissue types are to be avoided for treatment (e.g. used as a boundary for treatment), and/or when certain tissue types are to be treated (e.g. receive ablation energy). In some embodiments, tissue type data TTD comprises nerve location data, bone location data, blood vessel wall location data, and/or blood location data, such as when the identified tissue comprises tissue that are to be preserved (e.g. characterized as non-target tissue and not ablated). In some embodiments, system 10 (e.g. algorithm 215) is configured to provide a treatment plan using (e.g. based on an analysis of) two or more of: tissue type data TTD information; marked tissue information (e.g. tissue marked by device 100 as described herein, such as tissue identifiable by image analysis, elastography, and/or other analysis performed by algorithm 215); and/or clinician provided information (e.g. including clinician confirmation information). In some embodiments, algorithm 215 comprises an AI algorithm configured to perform on elastography analysis that differentiates treated from untreated tissue (e.g. and records the results in image data ID and/or registration data RD). In these embodiments, a treatment plan (e.g. as created by the clinician, system 10, or both) can safely include energy delivery to multiple targets, in a non-sequential manner, such as to avoid adversely effecting non-target tissue (e.g. the high temperatures that may result in sequentially treating multiple tissue targets in close proximity to one another). In some embodiments, algorithm 215 is configured to compensate for, and/or at least identify (e.g. and enter an alert mode), patient movement and/or undesired movement of device 100 (e.g. undesired movement of transmission assembly 1200 relative to the patient).

System 10 can be configured to produce a temperature map of target tissue to be treated, such as a temperature map included in image data ID and/or registration data RD that includes both target tissue locations as well as non-target tissue locations that are proximate the target tissue locations. The temperature map produced by system 10 can be produced during delivery of energy (e.g. in real-time), such as when the temperature map is used to adjust energy delivery by device 100, and/or to identify zones of tissue that have been ablated (e.g. versus zones that have not, as described herein). In some embodiments, imaging device 60 comprises an MRI and system 10 is configured (e.g. via algorithm 215) to produce a temperature map based on MR thermometry.

In some embodiments, system 10 is configured to deliver energy (e.g. ablative energy, marking energy, and/or other energy), to a first set of one or more particular volumes of target tissue using a first set of energy delivery settings, and to deliver energy (e.g. ablative energy, marking energy, and/or other energy) to a second set of one or more particular volumes of target tissue using a second set of energy delivery settings. The first and second sets of target tissue to be treated can include multiple discrete tissue targets, such as at least 5, 10, 25, and/or 50 tissue targets, and can include tissue types selected from the group consisting of: tissue of the tongue (e.g. as ablated in a sleep apnea treatment procedure); hair follicle or other hair segment tissue (e.g. as ablated in a hair removal procedure); tumor tissue (e.g. as ablated in a cancer or other tumor treatment procedure); prostate tissue (e.g. as ablated in a BPH procedure); and/or brain tissue (e.g. as treated in an epileptic focus or other brain tissue treatment procedure). In these embodiments, there can be differences in the first and second energy delivery settings, such as differences that are configured to compensate for differences in the target tissue (e.g. different volumes of target tissue, types of tissue in the target tissue) and/or to avoid damage to particular non-target tissue proximate the target tissue (e.g. to avoid adversely affecting nerves, blood vessels, and other potential non-target tissue). The differences in the first and second energy delivery settings can include one or more differences in: type of energy delivered (e.g. ultrasound energy versus electromagnetic, light, chemical, and/or other energy form); amplitude of energy delivery; frequency of energy delivery; waveform of energy delivery (e.g. waveform shape); duty cycle of energy delivery; modulation of energy delivery; steering of energy delivery; focusing of energy delivery; and combinations of these. In some embodiments, system 10 delivers HIFU, focused ultrasound, and/or other ultrasound energy to a first volume of tissue at a first frequency, and to a second volume of tissue, smaller than the first volume of tissue, at a second frequency that is higher than the first frequency. The higher frequency can be used to selectively avoid adversely affecting non-target tissue near the target tissue, such as nerves and/or blood vessels that are near the target tissue to be ablated.

In some embodiments, transmission assembly 1200 is configured to be positioned at multiple device locations DL, and to deliver energy (e.g. HIFU and/or other ultrasound energy) to at least one tissue target (e.g. hair segment) from each location DL. In some embodiments, system 10 is configured to monitor the positioning of transmission assembly 1200 (e.g. device 100) such as to create a map of all the locations DL, as well as a map of all the locations of tissue targets (treated and/or untreated). For example, functional elements 99, 199, 169, and/or 299 can comprise one or more position sensing elements (e.g. accelerometers, magnetic position sensors that are part of an electromagnetic positioning system, optical position sensors, proximity sensors such as tissue proximity sensors, and the like), such as to provide data that can be analyzed by algorithm 215 to record the position, and potentially the angular orientation, of device 100 (e.g. at all times during use). In some embodiments, algorithm 215 can be configured to produce a map of tissue targets to prevent undesired delivery of energy (e.g. delivery of energy to an already treated tissue target and/or to a recently treated tissue target, such as to avoid damage to non-target tissue, as described herein). In some embodiments, device 100 is configured to be robotically manipulated (e.g. by a functional element 99 comprising a robotic manipulator) to enable positioning at multiple device locations DL in order to deliver energy to a set of tissue targets that are located within a large tissue surface (e.g. the back or a leg of a patient). Alternatively or additionally, device 100 can be configured to be positioned at multiple locations manually by an operator (e.g. a clinician of the patient). In some embodiments, algorithm 215 comprises an artificial intelligence (AI) algorithm, such as an AI algorithm which is configured to efficiently deliver energy to multiple tissue targets from a single device location DL, and/or to determine a table of registration data RD to identify multiple device locations DL to which transmission assembly 1200 can be positioned (e.g. manually and/or robotically positioned). In these embodiments, algorithm 215 can utilize image data (e.g. as produced by transmission assembly 1200 and/or imaging device 60) comprising data produced via one or more imagining modalities, such as: ultrasound-based image data (e.g. as described herein); optical image data; magnetic resonance image data; x-ray image data; and combinations of these.

In some embodiments, all or a portion of device 100, and/or another component of system 10, is configured to be robotically manipulated by algorithm 215, such as when algorithm 215 comprises an AI algorithm configured to cause micromovements of transmission assembly 1200 during a tissue treatment and/or diagnostic procedure. In some embodiments, algorithm 215 is configured to cause transmission assembly 1200 to move based on an analysis of the anatomical location of non-target tissue (e.g. nerves) that are proximate to one or more tissue targets to be treated (e.g. to prevent damage to the non-target tissue). For example, during an ablative energy delivery, algorithm 215 can be configured to make small adjustments in transmission assembly 1200 position to avoid undesired tissue damage. In these embodiments, the identification of non-target tissue (e.g. as recorded in image data ID and/or registration data RD) can be: performed by a clinician; determined automatically by system 10 (e.g. via an AI-based algorithm 215); and/or identified via a combination of algorithm 215 identification and clinician confirmation. In some embodiments, algorithm 215 is configured to stop energy delivery if an undesired state is detected by algorithm 215, such as when algorithm 215 determines that ablative energy is being delivered to non-target tissue, and/or any circumstance in which non-target tissue is being adversely affected.

In some embodiments, system 10 is configured to treat a blood vessel (e.g. one or more varicose veins and/or spider veins) that are under a maximum diameter (e.g. vessels above the maximum are not treated), such as a maximum diameter of 2 mm, 1 mm, and/or 0.5 mm. In some embodiments, system 10 is configured to deliver energy (e.g. ultrasound energy) to treat a blood vessel, where the frequency of the energy delivered is based on the diameter of the segment of the blood vessel being treated, and/or the distance between the blood vessel segment and surface 1201 of transmission assembly 1200 (e.g. the distance between the blood vessel segment and elements 125).

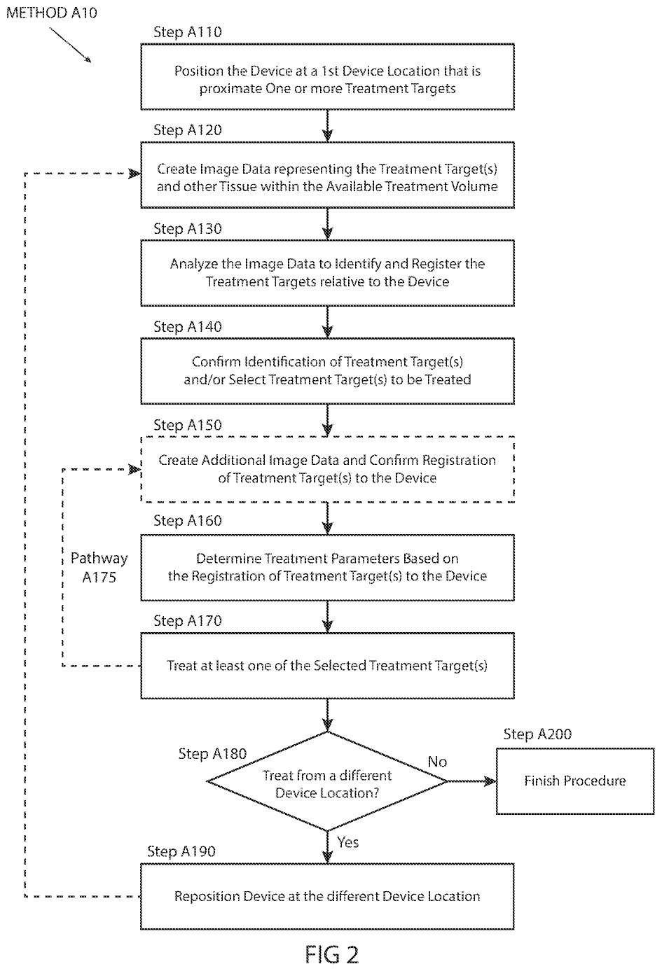

In some embodiments, system 10 is configured to treat target tissue as described in reference to FIG. 2 herein.

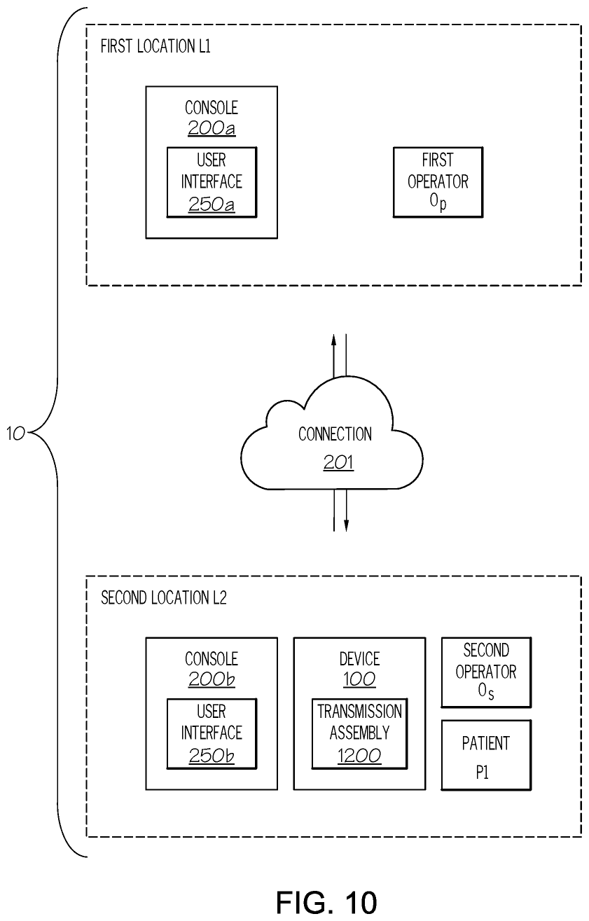

In some embodiments, system 10 is configured to allow an operator (e.g. a trained clinician or other qualified operator) to perform a medical procedure on a patient from a location that is remote from the patient, such as is described herein in reference to FIG. 10. For example, a qualified operator at a remote location, can assist another operator at the patient location. Confirmation of system 10 settings (e.g. energy delivery settings) and/or target tissue identification by a qualified operator (e.g. at a location remote from the patient) can be required (e.g. via algorithm 215) prior to treatment of the patient.

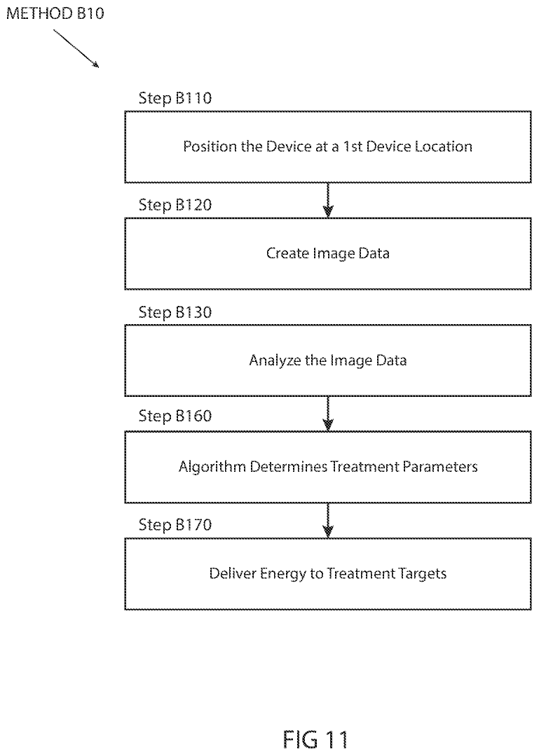

In some embodiments, system 10 is configured to vary the energy (e.g. ablative energy) delivered by transmission assembly 1200 based on the location of target tissue, such as is described herein in reference to FIG. 11. For example, system 10 can vary energy delivery (e.g. vary amplitude, frequency, focusing, and/or other energy delivery parameter) based on the relative distance between target tissue and non-target tissue.

Referring now to FIG. 2, a flow chart of a method of treating target tissue of a patient is illustrated consistent with the present inventive concepts. Method A10 of FIG. 2 is described using the various components of system 10 of FIG. 1 and otherwise herein.

In Step A110, device 100 (e.g. transmission assembly 1200) is positioned at a device location DL (e.g. location DL1 the first time Step A110 is performed), that is proximate one or more treatment targets. In some embodiments, transmission assembly 1200 is positioned on the skin of the patient.

In Step A120, image data ID (e.g. data ID1 the first time Step A120 is performed) is created representing the one or more treatment targets and other tissue within the available treatment volume ATV (e.g. volume ATV1 the first time Step A120 is performed). The image data ID can be created via transmission assembly 1200 (e.g. ultrasound imaging data), and/or via imaging device 60. In some embodiments, the image data ID represents data from both transmission assembly 1200 and imaging device 60. An algorithm 215 can be configured to differentiate target tissue from non-target tissue, such as a differentiation that can be confirmed by a clinician via a confirmation routine of algorithm 215 as described herein (e.g. confirmation from a qualified clinician at the patient location or at a location remote from the patient, as described in reference to FIG. 10 herein).

In Step A130, image data ID is analyzed to identify and register the treatment targets relative to transmission assembly 1200, creating registration data RD (e.g. data RD1 the first time step A130 is performed). The analysis can be performed manually, such as when the image data is presented as one or more images to an operator of system 10 (e.g. the patient's clinician) via user interface 250. Alternatively or additionally, the analysis can be performed by algorithm 215, such as in an automated or semi-automated arrangement. For example, algorithm 215 can identify one or more potential treatment targets and these targets can be presented to the operator to confirm or reject in Step A140.

In Step A140, the one or more treatment targets to be treated (e.g. to receive ablative energy) are confirmed (e.g. if already identified, such as via a confirmation routine of algorithm 215) or selected (e.g. if only a subset of total treatment targets identified in Step A130 are to be treated). Similar to Step A130, the confirmation and/or selection can be performed manually by an operator of system 10, and/or at least semi-automatically by algorithm 215.

In Step A150, an optional step of creating additional image data ID can be performed, such as image data ID representing the current set of treatment targets to be treated. The additional image data ID can be created via transmission assembly 1200 (e.g. ultrasound imaging data), and/or via imaging device 60. Based on the additional image data ID, the current registration data RD can be confirmed and/or adjusted as appropriate. System via algorithm 215, can be configured to detect undesired movement of transmission assembly 1200 (e.g. surface 1201) between steps, such as to cause system 10 to enter an alert state, and/or to cause system 10 to provide instructions to an operator, via display 2510 and/or another portion of user interface 250 on how to properly reposition transmission assembly 1200. In some embodiments, in Step A150, image data ID is created with a higher resolution proximate the identified treatment targets (e.g. a higher resolution than was created in step A120).

In Step A160, treatment parameters are determined based on the current registration data RD. For example, focusing of energy (e.g. ultrasound energy) at one or more tissue targets can be determined based on data RD. In some embodiments, safety margin tissue and/or non-target tissue is identified proximate the identified treatment targets. In some embodiments, one or more treatment parameters are selected to avoid damage to non-target tissue. In some embodiments, algorithm 215 is biased towards avoiding damage to non-target tissue. In some embodiments, algorithm 215 can determine a treatment target cannot be effectively treated without damaging surrounding non-target tissue and alert the operator. In some embodiments, the operator can select to ignore the alert and treat the target tissue.

In Step A170, the one or more selected (e.g. and confirmed) treatment targets are treated (e.g. receive ablative ultrasound or other energy from transmission assembly 1200 via transmission module 2200).

In some embodiments, after treating one or more treatment targets, a reconfirmation of proper positioning of transmission assembly 1200 is performed via reconfirmation pathway A175. In these embodiments, steps A150, A160, and A170 are repeated between target tissue treatments (e.g. between each successive treatment). In some embodiments, reconfirmation pathway A175 is performed within the time period in which a single tissue target is treated, for example, when reconfirmation pathway A175 is continuously performed as system 10 repeatedly switches between imaging mode and treatment mode during treatment of target tissue. In some embodiments, reconfirmation pathway A175 is performed if a change in position of device 100 is recognized by system 10, for example, when functional element 199 comprises an accelerometer and algorithm 215 detects a change in position of device 100 by analyzing data produced by functional element 199. In some embodiments, if a change in position is detected that is above a first displacement threshold (e.g. a portion of device 100, such as transmission assembly 1200, moves more than 1 mm) reconfirmation pathway A175 is performed. If a change in position is detected above a second threshold (e.g. a portion of device 100, such as transmission assembly 1200, moves more than 10 mm), method A10 can return to Step A110 (not shown), such that the operator must reposition device 100 (e.g. the operator must “start over” because device 100 moved too far to register the device to image data ID).

In Step A180, an option of treating from a different device location DL is considered. If a decision not to reposition (e.g. not to treat additional target tissue) is made, Step A200 is performed in which the procedure is completed. If repositioning to a different device location is to be performed (e.g. a device location DL2, DL3, DL4 as appropriate in repeating of the above steps), device 100 (e.g. transmission assembly 1200) is repositioned in Step A190, and method A10 returns to Step A120 and the procedure continues (e.g. defining a new available treatment volume ATV for each new device location DL and creating new registration data RD based on newly collected image data ID).

In some embodiments, algorithm 215 is configured to provide one or more treatment plans, as described herein, such as a treatment plan that can be confirmed and/or adjusted by the patient's clinician (e.g. the operator of system 10). System 10 can be configured to perform one or more steps of method A10 based on the treatment plan (e.g. a treatment plan determined after identification of one or more treatment targets, such as one or more treatment targets to be treated from one, two, or more device locations DL). In some embodiments, a treatment plan provided by algorithm 215 can comprise a first treatment comprising delivery of energy to one or more tissue targets, and a second treatment comprising delivery of energy to one or more similar and/or dissimilar tissue targets. The second treatment can be performed after a minimum time duration from the performance of the first treatment (e.g. from the end of the first treatment), such as a minimum time duration of at least 30 minutes, one hour, one day, one week, or one month. The minimum time duration between first and second treatments can be included to minimize swell of tissue (e.g. swelling of target tissue and/or non-target tissue).

In some embodiments, the one or more treatment targets comprise one or more hair segments that are to be treated (e.g. in a hair removal procedure as described herein). In these embodiments, system 10 can be configured to provide image data ID of the hair segments to be considered for treatment (e.g. eventual removal), such as to allow an operator to select each hair segment (e.g. hair bulb and/or other portion of the hair follicle) to be treated (e.g. ablated with ultrasound and/or other energy form). In some embodiments, multiple hair segments are identified for treatment within a single ATV (e.g. from a single device location DL).

In some embodiments, the one or more treatment targets treated in method A10 comprise one or more different types of tissue, as described herein. In some embodiments, the one or more targets treated include a non-tissue material, such as an implant (e.g. a tattoo pigment), a splinter, or other material embedded in and/or under the patient's skin, as described herein.

Referring now to FIGS. 3A, 3B, and 3C, various views of an ultrasonic assembly for creating image data and/or delivering treatment energy is illustrated consistent with the present inventive concepts. Transmission assembly 1200′ comprises a piezo-based ultrasound element, piezo substrate 1251. Assembly 1200′ further comprises a set of electrical conductors 1252a and 1252b (e.g. wires) that are positioned below and above (on the page) respectively, and in electrical contact with, piezo substrate 1251, creating an array of independently addressable piezo elements, piezo elements 1255, at each location where two wires intersect. Electrical conductors 1252a can be positioned orthogonal to conductors 1252b as shown. A second substrate, substrate 1253 (e.g. a substrate made of quartz) is positioned below and in contact with piezo substrate 1251 as shown. Substrate 1253 comprises a rectangular upper portion 1253a and a tapered lower portion 1253b, as shown. A matching layer, matching layer 1254, is positioned below and in contact with lower portion 1253b. The periphery of matching layer 1254 wraps around the bottom edges of lower portion 1253b, also as shown. The bottom surface of matching layer 1254, surface 1254E is configured to be positioned on tissue of a patient, such as to deliver and/or receive ultrasound energy, such as to create tissue image data and/or to deliver energy (e.g. ablation energy and/or other ultrasonic energy) to tissue.

Transmission assembly 1200′ (e.g. piezo substrate 1251) can comprise a cross-sectional area that is approximately four times the cross-sectional area of the available treatment volume ATV. For focusing at a depth of 5 mm below the tissue surface (e.g. the surface of the skin), substrate 1253 can comprise a thickness of 20 mm (e.g. to avoid multiple reflections). The angle of incidence of substrate 1253 (e.g. quartz) can be 45°, such that transmitted ultrasound will exit into the tissue at an angle of about 10°, correlating to an optimized F-number of three, and a width of 180 μm. The 45° incidence angle correlates to a thick substrate being offset 20 mm from the field of interest, thus requiring a 50 mm by overall area of the piezo elements 1255 to apply to a field of interest of 10 mm by 10 mm. In some embodiments, substrate 1253 comprises a material with a lower speed of sound than glass (e.g. having an angle of incidence less than 45°). In these embodiments, transmission assembly 1200′ (e.g. piezo substrate 1251) can comprise a cross-sectional area that is significantly less than four times the cross-sectional area of the available treatment volume ATV.

Matching layer 1254 enhances the ultrasound transmissions and reduces first and multiple reflections. Matching layer 1254 can also reduce power requirements of transmission assembly 1200′.

Transmission assembly 1200′ can be configured to transmit ultrasound energy at one or more frequencies, such as at a frequency between 20 MHz and 100 MHz. Transmission assembly 1200′ can be configured to perform beam forming of the ultrasound energy delivered by the array of piezo elements 1255.

Transmission assembly 1200′ can be configured as a two-dimensional (2D) array of piezo elements 1255, as described hereabove, each element 1255 addressable via an x-y addressing scheme provided by electrical conductors 1252a and 1252b (singly or collectively conductors 1252). Piezo substrate 1251 can comprise a piezoelectric material capable of operating in the 20 MHz to 100 MHz frequency range.

In the 25 MHz configuration of transmission assembly 1200′, the wavelength in a quartz-based substrate 1253 is 240 μm, which can correlate to an array of piezo elements 1255 having a 120 μm width and a 120 μm spacing (e.g. correlating to approximately 208 elements for a piezo substrate 1251 having a length and width of 50 mm).

As described herein, device 100 can use collected ultrasound data to form an image. In some embodiments, algorithm 215 comprises an AI algorithm configured to perform image reconstruction (e.g. without following traditional delay and sum beam forming methods).

The lateral dimensions of transmission assembly 1200 can be based on the size of the volume of tissue to be treated. For operating in tighter spaces around joints and curves in the skin, transmission assembly 1200 can be as small as 5 mm by 5 mm (e.g. to perform ultrasonic imaging and/or HIFU energy delivery). The dimensions of a piezoelectric of transmission assembly 1200 (e.g. a piezoelectric layer producing an array of piezo transducers, elements 125), such as its thickness, is determined by the desired frequency of operation, and the dimensions of the associated conductors (e.g. metal lines) and spaces between are dictated by the frequency of operation and the medium into which the piezoelectric is operating. The width of the conductors (e.g. metal lines) is about half a wavelength for a phase array operation. In some embodiments, transmission assembly 1200 comprises elements 125 comprising CMUT transducers in an array, and the dimensions of the array in terms of imaging can be the same as above.

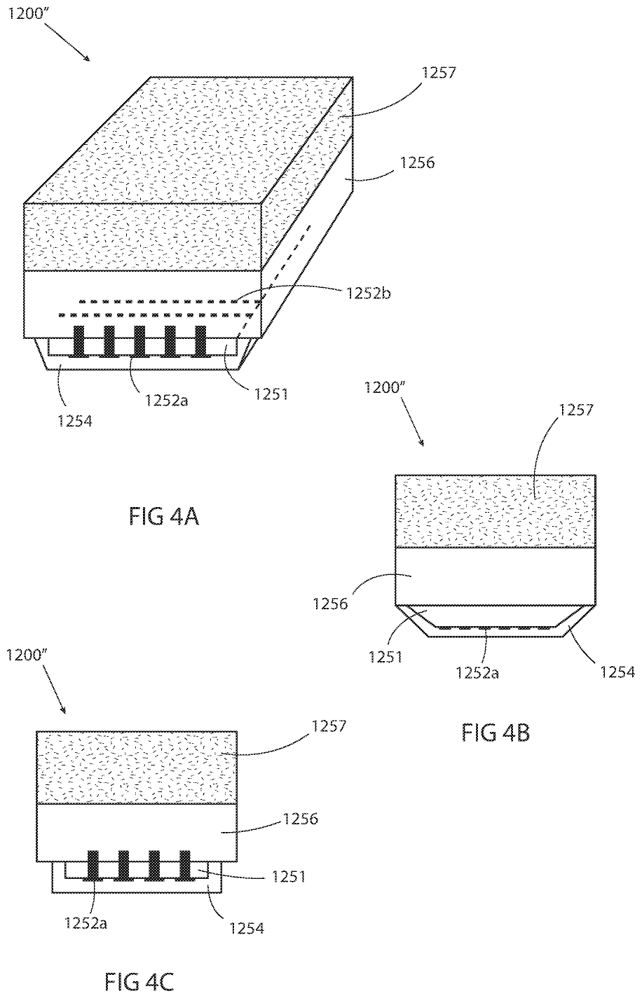

Referring now to FIGS. 4A, 4B, and 4C, various views of an ultrasonic assembly for creating image data and/or delivering treatment energy is illustrated consistent with the present inventive concepts. Transmission assembly 1200″ comprises a piezo-based ultrasound element, piezo substrate 1251. Assembly 1200″ further comprises a set of electrical conductors 1252a and 1252b (e.g. wires) that are positioned below and above (on the page) respectively, and in electrical contact with, piezo substrate 1251, creating at array of independently addressable piezo elements, piezo elements 1255, at each location where two wires intersect. Electrical conductors 1252a can be positioned orthogonal to conductors 1252b as shown. A second substrate, substrate 1256 (e.g. a substrate made of glass) is positioned above and in contact with piezo substrate 1251 as shown. A third substrate, substrate 1257 (e.g. comprising a composite of tungsten and epoxy) is positioned above substrate 1256, as shown. A matching layer, matching layer 1254, is positioned below and in contact with piezo substrate 1251. The periphery of matching layer 1254 wraps around the bottom edges of piezo substrate 1251, also as shown. The bottom surface of matching layer 1254, surface 1254E is configured to be positioned on tissue of a patient, such as to deliver and/or receive ultrasound energy, such as to create tissue image data and/or to deliver energy (e.g. ablation energy and/or other ultrasonic energy) to tissue.

Transmission assembly 1200″ can be configured as a 2D array with x-y addressing as described hereabove. Transmission assembly 1200″ can be configured to transmit ultrasound waves at a range of frequencies, such as at a frequency between 20 MHz and 100 MHz, such as between 25 MHz and 50 MHz.

Piezo substrate 1251 can comprise a 94 μm surface wave piezo material (e.g. a PZT material as manufactured by Murata), and substrate 1256 can comprise a glass material such as fused quartz. The upper layer of conductors 1252 can be positioned (e.g. deposited) on substrate 1256. Substrate 1257 can be made of a composite of tungsten and epoxy that is “lossy” (configured to efficiently dissipate energy passing therethrough), and of a matched impedance to dissipate energy transmitted from piezo elements 1255 that passes through substrate 1256 and into substrate 1257.

In the 25 MHz configuration of transmission assembly 1200″, the associated wavelength in tissue is 60 μm, which can correlate to an array of piezo elements 1255 having a 30 μm width and a 30 μm spacing (e.g. correlating to approximately 333 elements for a piezo substrate 1251 having a length and width of 20 mm).

Referring now to FIG. 5, a side view of a device for imaging and/or treating tissue and including an adjustable spacer is illustrated, consistent with the present inventive concepts. Device 100 of FIG. 5, and its components, can be of similar construction and arrangement as device 100 of FIG. 1 or otherwise described herein. Device 100 includes cable 151 as shown, which is configured to operably attach (e.g. electrically, sonically, fluidly, and/or mechanically attach) to console 200 of system 10. Device 100 can include a spacing device, spacer 160, which can be of similar construction and arrangement of spacer 160 described herein in reference to FIG. 1 and FIG. 6. Spacer 160 is configured to position surface 1201 of transmission assembly 1200 a particular distance, separation distance SD, from the tissue surface upon which device 100 has been placed, all as shown in FIG. 5. Distance SD can be chosen to allow focusing of energy delivered by transmission assembly 1200 (e.g. HIFU or other focused ultrasound energy) at a location separated from surface 1201 but on or relatively proximate the surface of the patient's skin. Spacer 160 can comprise a material that allows passage of energy therethrough (e.g. water or other fluid to allow passage of ultrasound energy therethrough). In some embodiments, spacer 160 surrounds a fluid, fluid 161 shown. Fluid 161 can comprise imaging agent 50 described in reference to FIG. 1 and otherwise herein.

Spacer 160 can be configured to have an adjustable thickness, such as to adjust the magnitude of distance SD (e.g. to accommodate a desired focusing distance associated with tissue targets on or proximate the patient's skin). For example, spacer 160 can comprise an accordion construction configured to expand as fluid 161 is introduced into spacer 160, and/or spacer 160 can comprise an expandable balloon configured to expand during fluid 161 introduction.

In some embodiments, device 100 includes functional element 199a shown, which can comprise a supply of fluid 161 and a pumping assembly. Functional assembly 199a can deliver fluid 161 to spacer 160, via tube 163 shown, to cause expansion of spacer 160 in direction D1 shown, and a corresponding increase in distance SD. Functional element 199a can be further configured to remove fluid 161, such as to cause contraction of spacer 160 (e.g. in the direction opposite D1) and an associated reduction in distance SD. In some embodiments, algorithm 215 described herein determines an optimal or other desired magnitude of distance SD (e.g. associated with an optimized or otherwise desired focusing distance of transmission assembly 1200), and algorithm 215 is configured to cause the automated delivery and/or removal of fluid from spacer 160, via operation of the pumping mechanism of functional element 199a, to correspondingly cause spacer 160 to expand and/or contract to achieve the desired magnitude of distance SD. In some embodiments, spacer 160 comprises a fluid access port, port 162 shown, such as a port that is penetrable by a needle and/or accessible via a syringe luer to allow fluid 161 to be delivered to and/or removed from spacer 160, such as to cause an associated expansion and/or contraction of spacer 160, respectively. Device 100 can include functional element 199a attached to tube 163, it can include port 162, or it can include both of these mechanisms configured to adjust the distance SD between surface 1201 and a tissue surface.

In some embodiments, fluid 161 is configured to cool device 100 (e.g. cool transmission assembly 1200) and/or to cool tissue that has been heated using device 100 (e.g. to avoid damage to non-target tissue). In these embodiments, device 100 can be configured to provide fluid 161 in a recirculating manner (e.g. provided in a recirculating manner by tube 163 and functional element 199a), such as to keep fluid 161 in spacer 160 at a temperature at or below body temperature of the patient. In some embodiments, functional element 199a is configured to cool fluid 161, such as when functional element 199a comprises a radiator configured to disperse heat and/or an active cooling element, such as a thermoelectric cooling element configured to cool fluid 161.

In some embodiments, spacer 160 comprises one or more coatings, coating 165 shown, such as an adhesive coating positioned on the skin-facing surface of spacer 160 and/or on the surface that faces transmission assembly 1200.

Spacer 160 can include one or more functional elements, such as functional element 169 shown. Functional element 169 can comprise one or more sensors, one or more transducers, and/or one or more other functional elements. In some embodiments, functional element 169 comprises a temperature sensor configured to provide a signal related to the temperature of spacer 160 and/or tissue proximate spacer 160, such as to prevent an undesired temperature from being reached (e.g. during energy delivery), such as when energy delivery is stopped when a threshold temperature is reached, and/or when energy is delivered in a closed-loop arrangement based on the measured temperature.

Referring now to FIGS. 5A-B, top and side sectional views of two types of spacers are illustrated, consistent with the present inventive concepts. Spacer 160 can include one or more portions, portions 160a shown, that are transmissive of the energy being delivered from and/or received by transmission assembly 1200, as well as one or more energy-blocking portions, portions 160b shown, that are configured to block or at least limit (“block” herein) the energy being delivered from and/or received by transmission assembly 1200. Portions 160a and 160b of spacer 160 can be geometrically arranged to direct energy (e.g. ultrasound energy) delivered by transmission assembly 1200 into specific portions of the patient's tissue (e.g. target tissue), while preventing or at least reducing (“preventing” herein) the delivery of the energy into other portions of the patient's tissue (e.g. non-target tissue). In some embodiments, energy delivery by transmission assembly 1200 is configured to avoid delivery into non-target tissue, and portion 160b is configured as a secondary measure of limiting damage to non-target tissue.

In FIG. 5A, spacer 160 comprises a single, inner transmissive portion 160a which is surrounded along its perimeter by a blocking portion 160b. As shown in the (bottom) sectional view of spacer 160 of FIG. 5A, blocking portion 160b can comprise a tapered cross-section, such as to focus (or assist in focusing) delivered energy to a desired (single) energy delivery zone as shown.

In FIG. 5B, spacer 160 comprises multiple inner transmissive portions 160a, each of which is surrounded along its perimeter by a blocking portion 160b. As shown in the (bottom) sectional view of spacer 160 of FIG. 5B, blocking portion 160b can comprise a tapered cross-section, such as to focus (or assist in focusing) delivered energy to associated desired energy delivery zones as shown. In some embodiments, spacer 160 comprises one or more portions configured to be selectively transmissive and/or blocking (e.g. by applying electric current to change the transmissive properties of a portion of spacer 160), such that algorithm 215 can pattern the transmissive and blocking portions of spacer 160 based on the area to be treated (e.g. based on analysis of image data ID). For example, algorithm 215 can be configured to pattern spacer 160 such that one or more portions of non-target tissue are protected by one or more blocking portions of spacer 160.

Referring now to FIG. 6, a top view of a skin-attached pad for serially locating a device for imaging and/or treating tissue at multiple device locations is illustrated, consistent with the present inventive concepts. Spacer 160 of FIG. 6 can be of similar construction and arrangement as spacer 160 of FIG. 1 or otherwise described herein. Spacer 160 can include a skin-compatible adhesive on its tissue contacting side. Spacer 160 of FIG. 6 includes a grid arrangement defining multiple locations, grid locations GL1-GL6 shown, at which surface 1201 can be positioned proximate the tissue underneath those grid locations GL. The geometry of spacer 160 can be configured (e.g. sized) to accommodate treatment of a skin surface of an equivalent size (e.g. equivalent length and width). The geometry of each individual grid location GL can be relatively the same as the geometry of surface 1201 (e.g. relatively the same length and width), such that surface 1201 “fits” with each grid location GL.

During a treatment procedure, device 100 can be serially positioned such that surface 1201 is at a first grid location GL, where imaging and/or a treatment step is performed, after which surface 1201 is moved to a second grid location GL, at which an imaging and/or treatment step is performed, and so on. In some embodiments, surface 1201 is positioned serially at multiple grid locations GL (e.g. GL1-GL6) to perform only an imaging procedure, after which surface 1201 is again positioned at these multiple grid locations (e.g. in the same order or a different order) to perform a treatment procedure (e.g. delivery of ablation or other energy). Each treatment procedure can include an imaging procedure as well (e.g. in order to confirm proper registration of the surface 1201). In some embodiments, algorithm 215 comprises an AI algorithm that determines the value for energy delivery parameters to be used at each grid location (e.g. for imaging and/or ablation), as well as the order for positioning in each grid location.

In some embodiments, spacer 160 comprises locating element 164 (twelve shown, two for each grid location GL). Locating element 164 can comprise an element configured to aid in the alignment or other registration of device 100 with each grid location GL. For example, one or more locating elements 164 can comprise a projection that is sized and arranged to mate with a corresponding hole in surface 1201 (e.g. one or more functional elements 199 comprise a mating hole in surface 1201). Alternatively or additionally, one or more locating elements 164 can comprise a magnet, which is configured to magnetically mate with a corresponding magnet of surface 1201 (e.g. one or more functional elements 199 comprise a magnet proximate surface 1201). Alternatively or additionally, locating element 164 can include an adhesive surface, such as to maintain contact between surface 1201 of device 100 and spacer 160. In some embodiments, spacer 160 is configured to visually change (e.g. change color) when a treatment is performed (e.g. ultrasound energy is delivered) through a portion of spacer 160 (e.g. at a grid location GL). For example, while treatment is performed at grid location GL1, colored (e.g. red) marks are created in spacer 160 in GL1. When device 100 is moved, the marks indicate to the operator which grid locations have been treated. In some embodiments, spacer 160 comprises a display, such as a flexible display comprising an OLED screen, that is adhesively applied to the skin of the patient. Algorithm 215 can generate a GUI (such as GUI 2511 not shown but described herein) to be displayed to the operator via spacer 160 comprising a display. The GUI can indicate to the user where treatment has been applied, and/or where to position device 100 to apply treatment.

Referring now to FIGS. 7A-G, various views of a CMUT transducer and its componentry are illustrated, consistent with the present inventive concepts. FIG. 7A shows orthogonal rows and columns of electrical conductors, such as conductors 1252a,b described herein. FIG. 7B shows one method of integrating a MEMS device (e.g. a CMUT array) onto an integrated circuit. The CMUT comprises through wafer interconnects (e.g. vias), and solder bumps connect the bottom of the via to the integrated circuit. FIG. 7C shows a sectional view of a CMUT assembly. A CMUT element is positioned on the top of the assembly, and a connection to the bottom electrode of the CMUT goes through the via conductor that is filled with polycrystalline silicone (polySi). On the bottom of the assembly, a solder ball connects the via to an imaging processing chip (e.g. an ASIC). FIG. 7D is a top view of a portion of a CMUT array. The array comprises multiple CMUT elements, and each element comprises CMUT cells (smaller circles shown) and vias (larger circles shown). Each element is 400 microns by 400 microns. A portion of a 2D array of 128 by 128 elements, each 400 microns on the side, is shown. FIG. 7E shows the bottom of the CMUT array of FIG. 7D. The via is shown in black. The polySi is the conductor that fills the via and covers part of the back of the array. The solder connects the polySi (the bottom of the CMUT) to the image processing chip. A metal test pad is added for probing the transducer before bonding to the image processing chip (e.g. in a manufacturing process). FIG. 7F shows a 16 by 16 2D array of CMUT elements. The left most two columns of the array are used to bring a ground contact to the back of the array. FIG. 7G shows a 128 by 128 array of CMUT elements on a 4 inch wafer.

Referring now to FIGS. 8A and 8B, side views of a flexible transmission assembly in flat and curved geometric states, respectively, are illustrated, consistent with the present inventive concepts. In FIG. 8A, transmission assembly 1200 comprises a housing portion 110a (e.g. a portion of housing 110 of device 100) that is constructed of flexible materials (such as a continuously flexible material or a series of rigid sections connected by flexible material). Flexibility of one or more portions of transmission assembly 1200, such as tissue contacting surface 1201, can be provided such as to allow transmission assembly 1200 to conform to a non-linear or otherwise non-flat contour of the patient's skin or other tissue (e.g. bending in one or more directions while maintaining contact with the patient's tissue). In some embodiments, spacer 160 is included, not shown but such as is described herein, between transmission assembly 1200 and the patient's tissue, and is configured to similarly flex with transmission assembly 1200 to match the contour of patient tissue.

An array of elements 125 (e.g. a 1D or 2D array of piezo and/or CMUT ultrasound elements) of transmission assembly 1200 of FIGS. 8A-B can be positioned on a substrate, substrate 126 shown, such as a substrate that is of similar or more flexibility as the flexibility of housing portion 110a. The elements 125 can be connected to wires, flex circuitry, and/or other drive circuitry, not shown but such as described herein. One or more sensor-based functional elements 199 can be positioned to provide one or more signals related to the current “geometric arrangement” (e.g. position and orientation) of one or more (e.g. all) of the elements 125 of the array (e.g. a straight, flat, geometry as shown in FIG. 8A, or a single or multi-planar curvilinear geometry as shown in FIG. 8B). A functional element 199 comprising one or more sensors can be positioned proximate (e.g. within and/or on) one or more portions of substrate 126, such as to provide signals related to the geometric arrangement of each of the elements 125 at all potential linear and curvilinear geometric states of transmission assembly 1200. In some embodiments, functional element 199 comprises one or more fiber bragg grating (FBG) sensors configured to provide signals related to the geometric arrangement of elements 125. In some embodiments, functional element 199 comprises one or more sensors selected from the group consisting of: optical sensors; strain gauges; magnetic sensors; and combinations of these, where the one or more sensors provide signals related to the geometric arrangement of elements 125.

As described herein, system 10 can be configured to deliver energy (e.g. ablative or other therapeutic energy) and/or receive energy (e.g. and produce an image based on the received energy) via signals provided to and/or received from, respectively, elements 125. The geometric arrangement of each of the elements 125 delivering and/or receiving energy can be used (e.g. by algorithm 215) to properly deliver energy and/or produce image data. In embodiments in which the geometric arrangement of elements 125 is fixed (e.g. housing portion 110a and/or substrate 126 is rigid), algorithm 215 can be configured to interpret received energy (e.g. to produce image data ID), and/or deliver energy (e.g. ablative or other therapeutic energy), based on the known, fixed arrangement. In the embodiments of FIGS. 8A-B, where the geometric arrangement of elements 125 is variable, algorithm 215 can be configured to determine the current geometric arrangement of elements 125 in order to interpret the received energy and/or delivered energy. In some embodiments, device 100 is configured to enter an alert state if algorithm 215 determines the geometric arrangement of elements 125 is outside of a functional threshold (e.g. too curved) and energy should not be delivered.

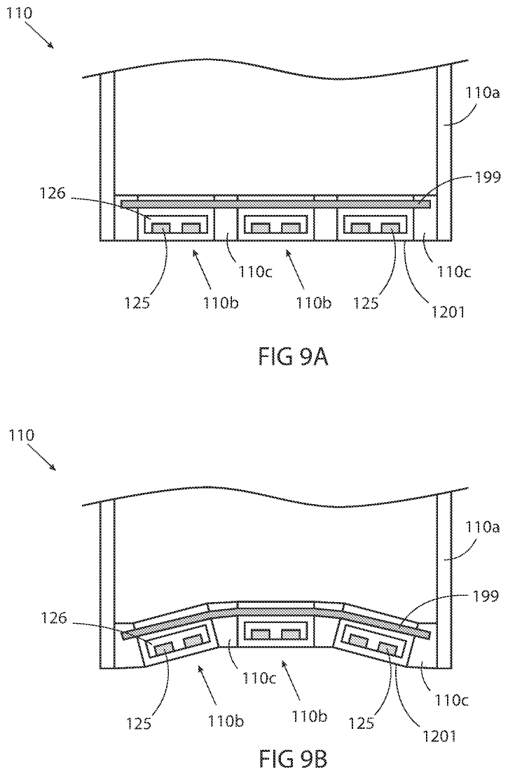

Referring now to FIGS. 9A and 9B, side views of a hinged transmission assembly in straight and curved geometric states, respectively, are illustrated, consistent with the present inventive concepts. In FIG. 9A, transmission assembly 1200 comprises a portion of housing 110 (e.g. a portion of housing 110 of device 100) that is constructed of multiple rigid portions 110b (three shown) that are connected by hinged portions 110c (two shown). Rotation of each rigid portion 110b relative to a neighboring rigid portion 110b allows transmission assembly 1200 to conform to a non-linear or otherwise non-flat contour of the patient's skin or other tissue (e.g. flexing in one or more directions while maintaining contact of surface 1201 with the patient's tissue). In some embodiments, spacer 160 is included, not shown but such as is described herein, between transmission assembly 1200 and the patient's tissue, and is configured to similarly flex with transmission assembly 1200 to match the contour of patient tissue.

An array of elements 125 (e.g. a 1D or 2D array of piezo and/or CMUT ultrasound elements) of transmission assembly 1200 of FIGS. 9A-B can be positioned on a substrate, substrate 126 shown, such as a substrate that is flexible (e.g. as described hereabove in reference to FIGS. 8A-B), and/or includes hinged portions similar to the hinged portions of housing 110 of FIGS. 9A-B. The elements 125 can be connected to wires, flex circuitry, and/or other drive circuitry, not shown but such as described herein. One or more sensor-based functional elements 199 can be positioned to provide one or more signals related to the current geometric arrangement of one or more (e.g. all) of the elements 125 of the array (e.g. a straight geometry as shown in FIG. 9A, or a rotated geometry as shown in FIG. 9B). In some embodiments, functional element 199 comprises one or more sensors (one shown) positioned along each of the hinged portions 110c, such as to provide a signal that can be used to determine the relative angle of rotation between the attached rigid portions 110b. In some embodiments, functional element 199 comprises one or more fiber bragg grating (FBG) sensors configured to provide signals related to the geometric arrangement of elements 125. In some embodiments, functional element 199 comprises one or more sensors selected from the group consisting of: optical sensors; strain gauges; magnetic sensors; and combinations of these, where the one or more sensors provide signals related to the geometric arrangement of elements 125.

A system 10 comprising the construction and arrangement of transmission assembly 1200 of FIGS. 9A-B can include an algorithm 215 that determines the geometric arrangement of each of the elements 125 (e.g. using the signals provided by one or more sensors of functional element 199), such as to use that geometric arrangement information to deliver energy and/or produce image data ID, as described hereabove in reference to FIGS. 8A-B.

In some embodiments, imaging device 60 comprises two or more optical cameras that provide images of a flexible portion of transmission assembly 1200, such as to be used by algorithm 215 to determine the geometric arrangement of elements 125.

Referring now to FIG. 10, a schematic view of a medical system configured to allow a qualified operator to perform a medical procedure from a remote location is illustrated, consistent with the present inventive concepts. System 10 of FIG. 10 includes a first console, console 200a, at a first location L1, and a second console, console 200b, at a second location L2. Consoles 200a and 200b are configured to transmit and receive information from the other, such as via a wired or wireless communication link, connection 201 shown, such as a cellular signal, a network such as the Internet, and the like. System 10 further includes device 100 at location L2. System 10 can be configured to allow a trained clinician or other qualified operator to oversee, guide, and/or otherwise control (“control” herein) a medical procedure of the present inventive concepts that is performed on a patient at a location that is remote from the qualified operator. As shown in FIG. 10, a first operator of system 10, primary operator O_{P} (e.g. one or more trained clinicians or other qualified operators) is at location L1. A second operator of system 10, secondary operator O_{S} (e.g. one or more nurses, technicians, and/or untrained clinicians) is located with the patient at location L2. In some embodiments, locations L1 and L2 are separated by a distance of no more than 500 ft, 250 ft, or 100 ft (e.g. locations L1 and L2 are two adjacent rooms or two rooms in the same building). In some embodiments, locations L1 and L2 are separated by a distance of at least 1 mile, such as at least 5 miles or at least 10 miles.

System 10 of FIG. 10, including device 100, console 200a, and console 200b, can include one or more components of similar construction and arrangement as similar components of system 10 described in reference to FIG. 1 and otherwise herein. Console 200a includes a user interface 250a, and console 200b includes user interface 250b, each as shown. Consoles 200a and console 200b are operably attached via connection 201 (e.g. to allow data transfer between the two). Console 200b is operably attached to device 100, such that signals (e.g. data signals and/or energy) can be transferred between console 200b and device 100 as described herein. Device 100 can comprise transmission assembly 1200 (e.g. for imaging and/or energy delivery) and other components of device 100 described herein.

In some embodiments, console 200b is configured to allow operator O_{S} to enter one or more system 10 settings (e.g. via user interface 250b), and algorithm 215 (e.g. an algorithm 215 included in either or both console 200a and 200b as shown) is configured to require confirmation of one or more of these operator O_{S} entered settings to be confirmed by operator O_{P} via console 200a (e.g. confirmation signals sent to console 200b via connection 201). For example, one or more energy delivery settings (e.g. as described herein), may require confirmation by operator O_{P} via console 200a prior to delivery of energy at that setting. System 10 can be configured to allow certain system settings to be set or at least confirmed by operator O_{P} via console 200a prior to their use by system 10, such as to cause a safe and effective procedure to be performed on patient P1 by a qualified operator that is at a location remote from the patient. Identification of one or more tissue targets (e.g. via algorithm 215 or otherwise as described herein) may require confirmation by operator O_{P} via console 200a prior to treatment (e.g. ablation and/or other energy delivery) of the one or more tissue targets.

In some embodiments, system 10 is configured to require that a treatment plan, as described herein, that is provided by a first operator (e.g. a qualified or non-qualified operator, such as operator O_{S} shown) must be confirmed by a qualified operator (e.g. a qualified operator, such as operator O_{P} at a remote location) prior to the implementation of that treatment plan by system 10.

In some embodiments, system 10 is configured to allow a qualified operator (e.g. operator O_{P} shown) at location L1, to perform a medical procedure on patient P1 at a remote location L2, without the need for any operator at location L2, or where the only operator at location L2 is patient P1 and/or a family member of the patient P1 (e.g. when a safe and effective procedure can be performed using system 10 solely with the control of a remote operator O_{P}). In these embodiments, location L2 can be the patient's home. In some embodiments, a single qualified operator (e.g. operator O_{P} shown) can oversee multiple second locations (e.g. multiple procedures, each being performed at a second location L2, by a second operator O_{S}, on a patient P) simultaneously. For example, via console 200a, operator O_{P} can provide confirmation input to multiple consoles 200b at various times (e.g. when required) during the multiple procedures being performed concurrently at various second locations L2.

Referring now to FIG. 11, a flow chart of a method for varying energy delivery settings based on the location of target tissue and/or non-target tissue is illustrated, consistent with the present inventive concepts. Method B10 of FIG. 11 is described using the various components of system 10 of FIG. 1 and otherwise herein. Method B10 may include similar steps as those described in reference to Method A10 of FIG. 2 described herein.

In Step B110, device 100 (e.g. transmission assembly 1200) is positioned at a device location DL (e.g. location DL1 the first time Step B110 is performed), that is proximate one or more treatment targets. In some embodiments, transmission assembly 1200 is positioned on the skin of the patient.

In Step B120, image data ID (e.g. data ID1 the first time Step B120 is performed) is created representing the one or more treatment targets and other tissue within the available treatment volume ATV (e.g. volume ATV1 the first time Step B120 is performed). The image data ID can be created via transmission assembly 1200 (e.g. ultrasound imaging data), and/or via imaging device 60. In some embodiments, the image data ID represents data from both transmission assembly 1200 and imaging device 60. An algorithm 215 can be configured to differentiate target tissue from non-target tissue, such as a differentiation that can be confirmed by a clinician via a confirmation routine of algorithm 215 as described herein (e.g. confirmation from a qualified clinician at the patient location or at a location remote from the patient, as described in reference to FIG. 10 herein).

In Step B130, image data ID can be analyzed, and treatment targets can be confirmed, such as is described in reference to Step A130 and A140, respectively, of FIG. 2. Additional image data can be created, such as to confirm registration of transmission assembly 1200, as described in reference to Step A150 of FIG. 2.

In Step B160, treatment parameters are determined based on the parameters of target tissue and/or non-target tissue. In some embodiments, the treatment parameters comprise energy delivery parameters (e.g. ultrasound energy delivery parameters) that are determined by algorithm 215. Algorithm 215 can comprise an AI-based or other algorithm that determines one or more energy delivery parameters based on one or more parameters selected from the group consisting of: distance between transmission assembly 1200 and target tissue (e.g. hair segments, nerves, tumor tissue, and/or other tissue to which delivery of ablative energy is desired); distance between transmission assembly 1200 and non-target tissue (e.g. nerves or other tissue to which damage is to be avoided); distance between target tissue and non-target tissue; and combinations of these. In some embodiments, transmission assembly 1200 is configured to deliver ultrasound energy, and algorithm 215 is configured to determine the frequency of ultrasound to be delivered based on the proximity of non-target tissue to target tissue (e.g. algorithm 215 increases delivery frequency the closer non-target tissue is to target tissue). In some embodiments, transmission assembly 1200 is configured to deliver ultrasound energy, and algorithm 215 is configured to determine the frequency of ultrasound to be delivered based on the proximity of non-target tissue to transmission assembly 1200 (e.g. algorithm 215 increases delivery frequency the closer non-target tissue is to transmission assembly 1200). In some embodiments, algorithm 215 is biased towards a higher frequency of ultrasound energy, such as to bias energy delivery away from deeper tissue locations.

In STEP B170, energy is delivered to one or more treatment targets with the energy delivery settings determined by algorithm 215 in Step B160.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A system for performing a medical procedure on a patient comprising:
a tissue interface device configured to image tissue of the patient and/or deliver energy to tissue of the patient, the tissue interface device comprising a transmission assembly; and
a console configured to transmit energy to the tissue interface device;
wherein the system is configured to perform a medical procedure on the patient.

2.-21. (canceled)
